(19) United States
(12) Patent Application Publication
(10) Pub. No.: US 2010/0304407 A1
(43) Pub. Date: Dec. 2, 2010

(54) Fluorescence Lifetime and Fluorescence Assays

(76) Inventors: Alexander Gray, Fife (GB); Michael John Paterson, Angus (GB); Dmitry Gakamsky, West Lothian (GB)

Correspondence Address: MYERS BIGEL SIBLEY & SAJOVEC, PO BOX 37428, RALEIGH, NC, 27627, US

(21) Appl. No.: 12/665,789

(22) PCT Filed: Jun. 20, 2008
(86) PCT No.: PCT/GB08/02134

§ 371 Date: Dec. 21, 2009

(30) Foreign Application Priority Data
Jun. 22, 2007 (GB) 0712109.8

(51) Int. Cl. C12Q 1/48; C12Q 1/42; G01N 33/58; G01N 21/64

(52) U.S. Cl. 435/7.72; 436/103; 435/15; 435/21

(57) ABSTRACT

The invention provides a method for determining a degree of phosphorylation of a substrate, for example a peptide substrate, using a fluorescence probe that acts alone or with another material and has a lifetime that varies when in proximity to a phosphate, the method comprising: causing the fluorescence probe to fluoresce; measuring a time response of the fluorescence, and analysing the fluorescence time response to identify a fluorescence component having a lifetime associated with phosphorylated substrate and a fluorescence component having a lifetime associated with un-phosphorylated substrate.

The present invention relates to the use of fluorescence lifetime techniques to determine peptide substrate phosphorylation. The invention also relates to the development of novel methods for use in assaying the activity of phosphatases and kinases. More particularly the invention relates to the measurement of fluorescence of fluorescently labelled substrates, in particular fluorescence lifetime, during assessment of phosphatases and kinases activity conducted in the presence of or after the reaction with a compound that selectively affects fluorescence of phosphorylated compounds and evaluating the data using a model based on the different fluorescence lifetimes of the probe for phosphorylated and un-phosphorylated substrates.

### Introduction

In recent years the phosphorylation and de-phosphorylation of proteins or lipids, catalysed by protein kinases and protein phosphatases, has been shown to be involved in the regulation of nearly all aspects of cell function. It has also become apparent that abnormalities of protein phosphorylation are involved in many diseases and pathological conditions, such as inflammatory diseases, cancer, diabetes, heart disease, and hypertension. This has led to an increase in interest in protein kinases and phosphatases as drug targets (P. Cohen, “Protein kinases—the major drug targets of the twenty-first century?” Nat Rev Drug Discov, 1(4), 309-15 (2002)).

Several protein kinase and protein phosphatase inhibitors are in clinical use at the moment. The immunosuppressant Cyclosporin, which inhibits the protein phosphatase 2B calcineurin, has found widespread use in the field of organ transplantation (J. Liu et al., “Calcineurin is a common target of cyclophilin-cyclosporin A and FKBP-FK506 complexes”, Cell, 66(4), 807-15 (1991)). An alternative immunosuppressant, rapamycin, inhibits the protein kinase mTOR (mammalian target of Rapamycin) (E. J. Brown et al., “A mammalian protein targeted by G1-arresting rapamycin-receptor complex”, Nature, 369(6483), 756-8 (1994)).

One of the first drugs to be developed by targeting of a specific protein kinase is Gleevec (STI 571), which is an inhibitor of the Abl tyrosine kinase. This drug has proven to be very effective in the treatment of chronic myelogenous leukaemia, a disease caused by conversion of the Abl kinase to a constitutively active form by a chromosomal rearrangement (M. J. Morin “From oncogene to drug: development of small molecule tyrosine kinase inhibitors as anti-tumor and anti-angiogenic agents” Oncogene, 19(56), 6574-83 (1994)). These clinical successes have greatly stimulated interest in protein kinases and phosphatases as potential drug targets.

Typically, one of the first steps in the drug discovery process after identification of a target enzyme involves screening compound libraries to identify ‘lead’ inhibitors of the enzyme, e.g. protein kinase, of interest. This requires that a suitable assay for the enzyme of interest is available. Traditional high-throughput screening (HTS) for protein-kinase inhibitors has made use of assays that measure the effects of compounds on the incorporation of radiolabelled phosphate into peptide and protein substrates. However, one of the major drawbacks of such radioactive assay formats is that, due to the large numbers of compounds present in these libraries (typically >1 million), large quantities of radioactive materials are required to run them. Moreover, safety and environmental considerations involved in the storage and/or disposal of radioactive waste have highlighted the need for the development of alternative non-radioactive assay formats.

Several non-radioactive assay formats are available. These include an ELISA type assay based on an antibody specific for a common epitope in a set of generic substrate peptides (H. Ross, C. G. Armstrong and P. Cohen “A non-radioactive method for the assay of many serine/threonine-specific protein kinases”, Biochem J, 366(Pt 3), 977-81 (2002)). This has been further adapted using fluorescently labelled peptides in a fluorescence polarization (FP) assay. Other formats are also available based on anti phosphotyrosine antibodies using FP and fluorescence intensity. Other available assays either rely on antibodies (generic or specific), fluorescence intensity, fluorescence polarization or in some cases fluorescence resonance energy transfer (FRET). All fluorescence intensity assays, based on steady-state detection of the emitted light using a continuous light source, are susceptible to interference emission by the compounds being screened resulting in high rates of false positive and negative results, as well as some methods are also unsuitable for HTS applications due to the number of washing steps involved in the assay method.

All fluorescence intensity assays are based on steady-state detection of the emitted light using a continuous light source. These methods are susceptible to interfering emission by test compounds in the chemical libraries being screened resulting in high rates of false positive and negative results. Another disadvantage of the intensity-based methods is that some of them require multiple washing/separating steps, which are unsuitable for HTS applications.

As an alternative to fluorescence intensity based methods, one based on fluorescence lifetime has been proposed, as described in WO03/089665, which teaches techniques for applying the measurement of fluorescence lifetime to a specific class of protein kinases and phosphatases. These are not, however, applicable for investigating the phosphorylation and de-phosphorylation of other classes of protein or lipid kinase and phosphotase.

The lifetime, τ, of a fluorescence probe is referred to the time the molecule ‘lives’ in its excited state before emitting a photon. If fluorescence obeys a first-order kinetic mechanism its intensity decays exponentially according to:

I(t)=I₀e^{-t/τ} (1)

Lifetime relates to the time for the fluorescence intensity to decay to 1/e or 36.7% of the original intensity. The value of this lifetime for a typical fluorescence probe is in the sub-nanosecond to tens of nanosecond range and is a function of its chemical structure, which can be affected by the environment of the probe including the proximity of quenching or fluorescence enhancing reagents. Fluorescence lifetime measurements can be based either on the excitation of the fluorophore by short, usually of pico- or nano-second duration, optical pulses and monitoring the time response of the emission or using a frequency modulation technique (cf. Principles of Fluorescence Spectroscopy, ed. J. R. Lakowicz, Second Edition, Kluwer, Academic Press, 1999) or any other method providing information about the rate of depopulation of the excited state.

The present invention relates to an improved, and more generally applicable, method for using fluorescent lifetime measurements to provide an indication of the level of phosphorylation in, for example, a peptide substrate.

### SUMMARY OF THE INVENTION

The present invention involves analysing the fluorescence time response of a fluorescence probe labelled substrate in terms of the lifetimes for phosphorylated and un-phosphorylated states to give a quantitative determination of the level of phosphorylation. Ideally, the probe should have a sufficiently long fluorescence lifetime to enable differentiation from interfering emissions, typically in a few nanoseconds time-domain and be independent of sample pH or temperature. The present invention is based on the notion that the overall fluorescence time response of the labelled substrate comprises at least two components having two different lifetimes, one due to fluorescence of the probe in the absence of phosphate group in the substrate and one due to fluorescence of the probe in the presence of the phosphate group. By decomposing these two components from the total fluorescence response, there is provided a simple and effective method for quantitative determination of the relative proportion of the phosphorylated and un-phosphorylated substrates. Each of these two components could be composed of two or more exponential terms, and the method could be extended to decompose more than two components.

According to a first aspect of the invention, there is provided a method for determining the level of phosphorylation/dephosphorylation of a substrate, the method comprising:
using a fluorescence probe conjugated to a substrate that acts alone or in conjunction with another material and has a lifetime that varies when in proximity to a phosphate group;
causing the fluorescence probe to fluoresce;
measuring a time response of the fluorescence, and
analysing the fluorescence time response to identify a fluorescence component having a lifetime associated with phosphorylated substrate and a fluorescence component having a lifetime associated with un-phosphorylated substrate.

Using the method of the invention, the relative concentrations of the phosphorylated and un-phosphorylated fractions can be determined. This can be done using the fluorescence time response, I(t), of the sample and evaluating the data using a model function constructed as a linear combination of emission functions of phosphorylated, I₁(t), and unphosphorylated, I₂(t) substrates:

I(t)=A₁I₁(t)+A₂I₂(t) (2)

where A₁ and A₂ are amplitude coefficients of phosphorylated and unphosphorylated substrates respectively. Hence, the fraction of unphosphorylated substrates, F₂, is given by:

F₂=A₂/(A₁+A₂) (3)

The I₁(t) and I₂(t) functions can be determined by measuring the emission of control samples, i.e. the fluorescence from samples containing the phosphorylated and unphosphorylated substrates only.

Fitting of the fluorescence time response to this model allows quantitative determination of the phosphorylated and unphosphorylated substrate concentrations and provides an unambiguous mechanism for discrimination of failed assays based on the quality of the fit to the model (equation (2)). The criteria for the fit quality include, for example, the normalised statistical goodness of fit parameter, X², which is ideally close to 1, and the random distribution of the weighted residuals. An unsatisfactory fit indicates the presence of an additional (interfering) emission of the test compound superimposed on the emission of the probe. Samples exhibiting unsatisfactory fits could be considered as a potential source of false hits and should be assessed using different experimental conditions, for example using either a different emission wavelength where the impact of the interfering emission is not significant or using an orthogonal method.

Using fluorescence lifetime measurements offers significant advantages compared with established fluorescence intensity measurements, as the fluorescence lifetime is independent of many experimental parameters, such as sample concentration and volume, excitation and emission wavelength, excitation intensity and experimental geometry. Further improvements can be achieved by using time resolved fluorescence measurements, as these allow emissions from different species to be separated based on differences in their fluorescence lifetime. This allows the probe emission to be differentiated from emission from different interfering compounds, including emission of screening compounds, thereby to reduce the number of false positive or negative hits present in the fluorescence intensity assays.

To produce a selective change in the fluorescence lifetime of the probe if the substrate is phosphorylated, a ligand compound may be used. This may be selected to interact with both the phosphate group and the fluorescence probe of the substrate to produce a ternary complex, whilst preferably not affecting the lifetime of the probe if the substrate is un-phosphorylated. Suitable selection of the ligand can provide a high affinity ternary complex allowing a relatively low concentration of the ligand to be used, thereby limiting possible effects on the emission of the probe on the un-phosphorylated substrate by the non-specific collision mechanism. Using a suitable ligand compound provides the opportunity for there to be a considerable difference in the fluorescence lifetime of the probe dependent on the phosphorylation state of the substrate to allow the fluorescence components to be separated by the model-based analysis.

Examples of ligands that have been demonstrated include phenylmalonic acid/iron(III), 2-hydroxyacetophenone/iron(III) or Bovine Serum Albumin derivatised with complexes of diethylene triamine pentaacetic acid/iron(III).

The method of the invention can be used in a variety of formats. For example for a cell-free solution-phase assay, the method may involved reacting kinase or phosphatase with an appropriate substrate labelled with a fluorescence probe; introducing a ligand to the reaction mixture, wherein the ligand affects the fluorescence time response of the probe; measuring the resultant fluorescence time response; evaluating the time response using the model (Equation 2); and determining the A₁ and A₂ coefficients, thereby to determine the concentrations of the phosphorylated and un-phosphorylated substrate fractions.

Another solution-based assay format involves detection of a change in phosphorylation of non-labelled substrate, mediated by either a protein kinase or a phosphatase, by competition for binding to a high molecular weight ligand that has been complexed with fluorescently labelled phosphorylated peptide. In a kinase assay the resultant competition for binding to the ligand by non fluorescent phosphorylated substrates results in displacement of the fluorescently labelled peptide from the complex with a concomitant change fluorescence lifetime. An example of this is shown schematically in FIG. 32 in which the high molecular weight ligand complex is based upon BSA. In a phosphatase assay the unlabelled substrate would already be phosphorylated and in competition for ligand binding therefore action of the phophatase is measured by the degree of increasing association of the labelled peptide with the ligand caused by removal of the competing phosphorylated peptide. In this assay format quantitation may be by comparison to a standard displacement curve using known amounts of unlabelled phosphorylated peptide in competition with a set amount of ligand complex.

Yet another option exists for a cell based assay involving the introduction of a labelled substrate into cells; allowing phosphorylation/de-phosphorylation of the substrate by intracellular enzymes; lysing the cells; adding a ligand to the cell lysate; measuring the resultant fluorescence time response; evaluating the data using the model (Equation 2) and determining the A₁ and A₂ coefficients, thereby to determine the concentrations of the phosphorylated and un-phosphorylated substrate fractions.

According to a second aspect of the invention, there is provided a system for determining phosphorylation of a substrate, preferably a peptide substrate, using a fluorescence probe that either acting alone or in conjunction with another material and has a lifetime that varies when in proximity to phosphate, the probe being applied in use to the substrate, the system comprising:
means for causing the fluorescence probe on the substrate to fluoresce;
means for measuring a time response of the fluorescence, and
means for analysing the fluorescence time response to identify fluorescence having a lifetime associated with phosphorylated substrate and fluorescence having a lifetime associated with un-phosphorylated substrate.

According to a third aspect of the invention, there is provided a computer program or computer program product, for determining substrate phosphorylation using a measured fluorescence time response of a fluorescence probe that either acting alone or in conjunction with another material has a lifetime that varies when in proximity to phosphate, the probe being applied in use to the substrate, the computer program preferably being on a computer readable medium or data carrier and having code or instructions for analysing the fluorescence time response to identify fluorescence having a lifetime associated with phosphorylated substrate and fluorescence having a lifetime associated with un-phosphorylated substrate.

In addition to the highly advantageous use of fluorescence lifetime techniques for determining the concentrations of phosphorylated and un-phosphorylated substrate fractions, it has been surprisingly found that certain quenching moieties may be used in assays for measuring kinase and phosphatase activities upon substrates or potential substrates and/or in the presence of inhibitors or potential inhibitors. The quenching moieties are brought into proximity with a fluorophore if the fluorophore is in proximity with a phosphate moiety. When employed in such assays, therefore, the difference between the fluorescence, in particular the fluorescence lifetime, of substrates and products is enhanced, improving the accuracy of these assays, and allowing the detection of small amounts of reaction, as is convenient, for example, when measuring initial rates of enzyme activity.

Viewed from a fourth aspect, therefore, the invention provides a method for investigating the phosphorylation of a fluorescently labelled test compound by a kinase, said method comprising contacting said test compound with said kinase and a quenching moiety and measuring the resultant fluorescence, said quenching moiety quenching fluorescence from the test compound upon phosphorylation of the test compound.

Viewed from a fifth aspect, the invention provides a method for investigating the dephosphorylation of a fluorescently labelled test compound by a phosphatase, wherein said test compound comprises a phosphate moiety, said method comprising contacting said test compound with said phosphatase and a quenching moiety and measuring the resultant fluorescence, said quenching moiety quenching fluorescence from the test compound.

Viewed from a sixth aspect the invention provides a kit of parts suitable for use in a method according to the fourth or fifth aspects of this invention comprising a fluorescently labelled kinase or phosphatase substrate and a quenching moiety that quenches fluorescence of the phosphorylated test compound.

Viewed from a seventh aspect, the invention provides a method for investigating the activity of a kinase, said method comprising

(i) exposing the kinase to a test compound in the presence of a complex comprising a fluorescently labelled phosphorylated compound and a quenching moiety, for example a protein, wherein said quenching moiety quenches fluorescence of the fluorescent label in said complex; and
(ii) measuring the resultant fluorescence.

Viewed from an eighth aspect, the invention provides a method for investigating the activity of phosphatase, said method comprising

(i) exposing the phosphatase to a test compound wherein some of said test compound is present in a complex comprising said test compound and a quenching moiety, for example a protein, in the presence of a fluorescently labelled phosphorylated compound wherein said quenching moiety quenches fluorescence of the fluorescent label upon complexation of the quenching moiety to the fluorescently labelled phosphorylated compound; and
(ii) measuring the resultant fluorescence.

Viewed from a ninth aspect, the invention provides a kit of parts suitable for use in a method according to the seventh or eighth aspects of this invention comprising a fluorescently labelled phosphorylated compound and quenching moiety, for example a protein, with which it may be complexed.

The use of fluorescent labels or probes permits the determination of fluorescence (e.g. fluorescence intensity or fluorescent lifetime, typically fluorescence lifetime, or other fluorescence parameters) as is described in greater detail hereinafter. Any convenient fluorescent label or probe may be used.

Further aspects of the embodiments of the present invention will be evident from the discussion that follows.

### BRIEF DESCRIPTION OF THE FIGURES

Various aspect of the invention will now be described by way of example only and with reference to the accompanying drawings, of which:

FIG. 1 is a schematic diagram of a fluorescence lifetime spectrometer;

FIG. 2 is a schematic illustration of a lifetime-based assay;

FIG. 3 shows AA9 emission time-courses in the presence of increasing concentrations of the quencher (left panel);

FIG. 4 is another schematic illustration of a lifetime-based assay;

FIG. 5 shows a family of fluorescence time-courses of mixtures of ACE14-labelled phosphorylated and unphosphorylated substrates containing gradually decreasing from 100 to 0 proportion of the phosphorylated peptide in the presence of 2.5 mM PMA/Fe³⁺ quencher;

FIG. 6 shows normalized amplitudes of the quenched and unquenched components, A₁/(A₁+A₂)*100% and A₂/(A₁+A₂)*100%, for the different proportions peptide mixtures of FIG. 5;

FIG. 7 shows the normalised amplitudes for the quenched and unquenched components, A₁/(A₁+A₂)*100% and A₂/(A₁+A₂)*100% as a function of phosphorylated peptide;

FIG. 8 shows further examples of the relative quality of intensity, average lifetime, to the use of a photophysical model and FAST software;

FIG. 9 shows the results of a radiometric assay in which ACE 14-labelled () and unlabelled (∘) Crosstide synthetic substrate is phosphorylated;

FIG. 10 shows the effect of peptide phosphorylation on fluorescence lifetime with three concentrations of labelled Crosstide peptide (30, 20 and 10 μM) and with controls (Cont) in which the assays were conducted with boiled enzyme;

FIG. 11 shows fluorescence lifetime decay curves for PKB assays conducted with a series of synthetic phosphorylated ACE 14-labelled peptides in which the label is at different distance from the phosphate in each member of the series;

FIG. 12 shows fluorescence lifetime decay curves of compounds [1] and [6] from Example 3 conducted in the presence and absence of ferrous sulphate;

FIG. 13 shows a plot of fluorescence lifetime against concentration of a chelate formed between iron(III) and phenylmalonic acid (PMA) in the presence of both phosphorylated and unphosphorylated peptide;

FIGS. 14A and 14B each show fluorescence lifetime decay curves of the 6 ACE 14-labelled peptides described in Example 3 in the absence (FIG. 14A) and presence (FIG. 14B) of iron (III) PMA chelate;

FIG. 15 shows a plot of the fluorescence lifetime against distance from the phosphorylated residue (FIG. 15) in respect of the 6 ACE 14-labelled peptides described in Example 3 (distance 6 corresponds to the control unphosphorylated peptide [1]);

FIG. 16 shows a plot of the fluorescence lifetime against the proportion of phosphopeptide [6] in mixtures of phosphopeptide [6] and control unphosphorylated peptide [1], at different concentrations of PMA Fe(III) chelate;

FIG. 17A shows a plot of fluorescence lifetime against units of PKB after 40 minutes for an assay conducted using ACE 14-labelled Crosstide peptide; FIG. 17B shows the dependency of fluorescence lifetime against time for the same assay conducted in the presence and absence of PKB;

FIG. 18 shows a plot of fluorescence lifetime for seven groups of 4 experimental and four control data points, the controls being carried out using boiled PKB, other parameters being as described for Experiment 8;

FIG. 19A shows a plot of fluorescence lifetime against units of MAPKAP K2 after 40 minutes for an assay conducted using N-acetylated KLNRTLSVA with the label on the lysine side chain; FIG. 19B shows the dependency of fluorescence lifetime against time for the same assay conducted in the presence and absence of MAPKAP K2;

FIG. 20 shows a plot of fluorescence lifetime against units of SGK1 after 40 minutes incubation for an assay conducted using ACE 14-labelled Crosstide as substrate;

FIG. 21 shows a plot of fluorescence polarisation against increasing amounts of two pairs of labelled phosphorylated and unphosphorylated peptides in the presence of a BSA iron chelate;

FIG. 22 shows a plot of fluorescence lifetime against increasing amounts of two pairs of labelled phosphorylated and unphosphorylated peptides in the presence of a BSA iron chelate;

FIG. 23 shows a plot of fluorescence lifetime against increasing concentrations of a phosphorylated hexapeptide in the presence of a BSA iron (III) chelate complexed with 9-aminoacridone labelled Crosstide;

FIG. 24 shows plots of fluorescence lifetimes against increasing concentrations of a phosphorylated hexapeptide in the presence of four different concentrations of BSA iron (III) chelate complexed with 9-aminoacridone labelled Crosstide;

FIG. 25 demonstrates the efficacy of the assay in a screening situation in which a comparison of a radiometric PKB assay and fluorescence lifetime assay was carried out using a compound series sent for screening by a pharmaceutical company. In FIG. 25A underlined data denotes a significant inhibition of activity scored as a hit; FIG. 25B shows that all hits detected by the radiometric assay are also detected by the lifetime assay;

FIG. 26 shows the mechanism of an Invitrogen assay;

FIG. 27 shows the structure of SOX and the principle of its fluorescence modulation;

FIG. 28 shows a family of fluorescence time-courses of phosphorylated and unphosphorylated substrates containing 25 mM MgCl₂;

FIG. 29 shows concentration dependences of the fast and slow fluorescence component fractions, f₁=A₁/(A₁+A₂) and f₂=A₂/(A₁+A₂), as a function of the percentage of phosphorylation for the data of FIG. 28;

FIG. 30 shows a family of fluorescence time-courses of phosphorylated and unphosphorylated substrates containing 15 mM MgCl₂;

FIG. 31 shows concentration dependences of the fast and slow fluorescence component fractions, f₁=A₁/(A₁+A₂) and f₂=A₂/(A₁+A₂) as a function of the percentage of phosphorylation for the data of FIG. 30, and

FIG. 32 shows in schematic form a way in which the seventh aspect of the invention may be practised.

FIG. 33 shows a plot of initial rates of reaction of phosphorylation (in μmol/min) of a generic peptide by the kinase MSK-1 against the concentration of a peptide (in μM) in accordance with a method of the invention, and as described in Example 18.

FIG. 34 shows a plot of initial rates of reaction of phosphorylation (in μmol/min) of a generic peptide by the kinase PKA against the concentration of a peptide (in μM) in accordance with a method of the invention, and as described in Example 18.

FIG. 35 shows a plot of initial rates of reaction of phosphorylation (in μmol/min) of a generic peptide by the kinase PRAK1 against the concentration of a peptide (in μM) in accordance with a method of the invention, and as described in Example 18.

FIG. 36 shows a comparison of inhibition activities of a common set of test compounds assayed as inhibitors of PKA, with differences between the data of % activity of PKA in the presence of the test compounds between the two assays indicated by the differences in shadings/presentations of the numbers, as is explained in Experiment 19.

FIG. 37 shows a comparison of inhibition activities of a common set of test compounds assayed as inhibitors of PKB, with differences between the data of % activity of PKB in the presence of the test compounds between the two assays indicated by the differences in shadings/presentations of the numbers, as is explained in Experiment 19.

### DETAILED DESCRIPTION OF THE INVENTION

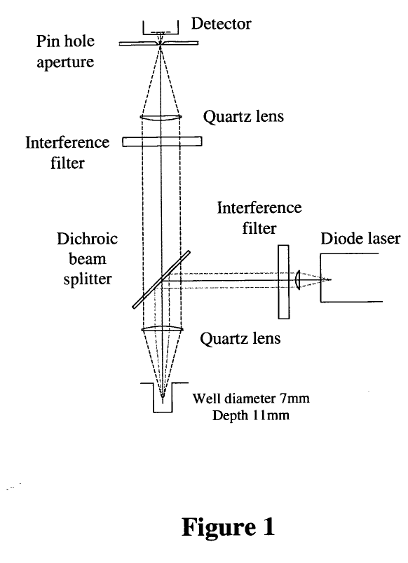

FIG. 1 shows a typical optical system for measuring fluorescence lifetime for emissions from a sample in a plate reader. This has a picosecond diode laser for emitting light at a wavelength suitable for stimulating sample fluorescence, an interference filter in front of the laser for removing unwanted wavelengths, a dichroic beam splitter for directing filtered light from the laser towards a well of the plate reader, and a focusing lens for focusing light into the sample well, and so onto a sample contained therein. Fluorescent light emitted by the sample, at a shifted wavelength, in response to excitation by the laser follows a collection path to a detector. On the collection path is the focusing lens, the dichroic beam splitter and a collector arrangement. The beam splitter is transparent at the wavelength of the fluoresced light so that such light passes through it and along the collection path onto the detector. The collector has in sequence an interference filter and a collection lens for focusing the fluoresced light onto the detector. The detected output is processed, for example using time correlated single photon counting electronics and data processing techniques are then used to determine the fluorescence lifetime and analyse the chemical and biochemical samples.

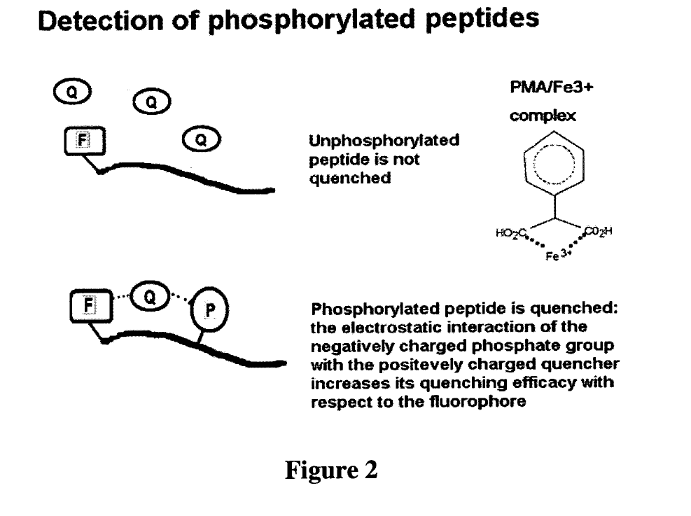

In accordance with the invention, there is provided a method for measuring changes in fluorescence lifetime of a probe conjugated to a peptide substrate due to its phosphorylation/dephosphorylation. In order to influence fluorescence emission of the probe attached to the phosphorylated substrate (peptide) a selective fluorescence quencher is used. This is illustrated in FIG. 2. In this specific example, the quencher is composed of two functional parts. One is positively charged and can selectively bind to the peptide due to the electrostatic interaction with the phosphate group which caries a negative charge. The other part provides fluorescence quenching by interaction with the probe when the quencher is bound to the substrate. In a preferred example, a complex of phenylmalonic acid with Fe³⁺ ion is used as the quencher.

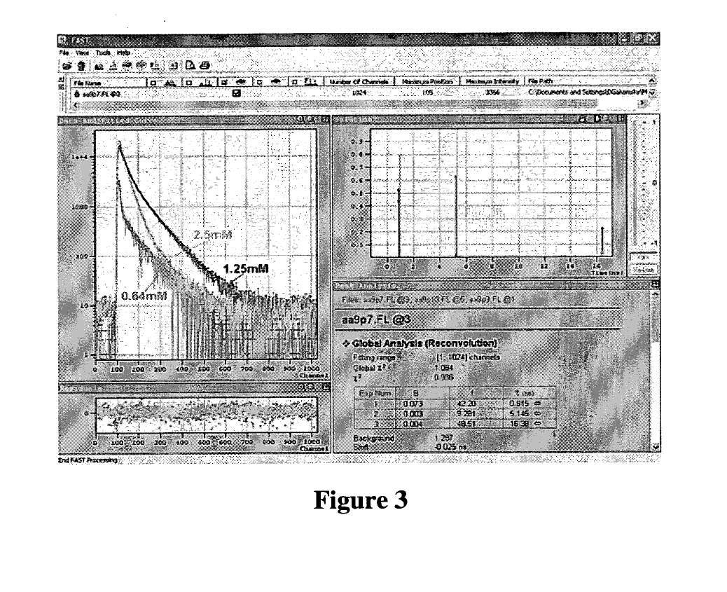

Binding of the quencher to the phosphate group leads to creation of a complex between the latter, the quencher and the fluorophore. The interaction of the quencher's organic moiety with the fluorophore significantly changes its lifetime. One aspect of the invention resides in the unexpected realisation that there are three different states, a high affinity state in which strong quenching is observed, a low affinity state in which weak quenching is observed and a state in which there is no quenching, as illustrated in FIG. 3. In each of these states, the fluorescence lifetime of the fluorophore is different and this difference can be readily detected. Hence, a mixture of phosphorylated and unphosphorylated substrate can be described by a function having three exponential components, the first two exponential components being due to the quenched emission of the strongly and weakly quenched fluorophore and the last component being due to the unquenched emission from the unphosphorylated substrate. Due to a relatively high affinity of the quencher for the phosphate group quenching of the fluorescence probe on the unphosphorylated substrate due to diffusion can be neglected. Hence changes in the fluorescence lifetime of the fluorophore can be attributed to quenching induced by the phosphate group.

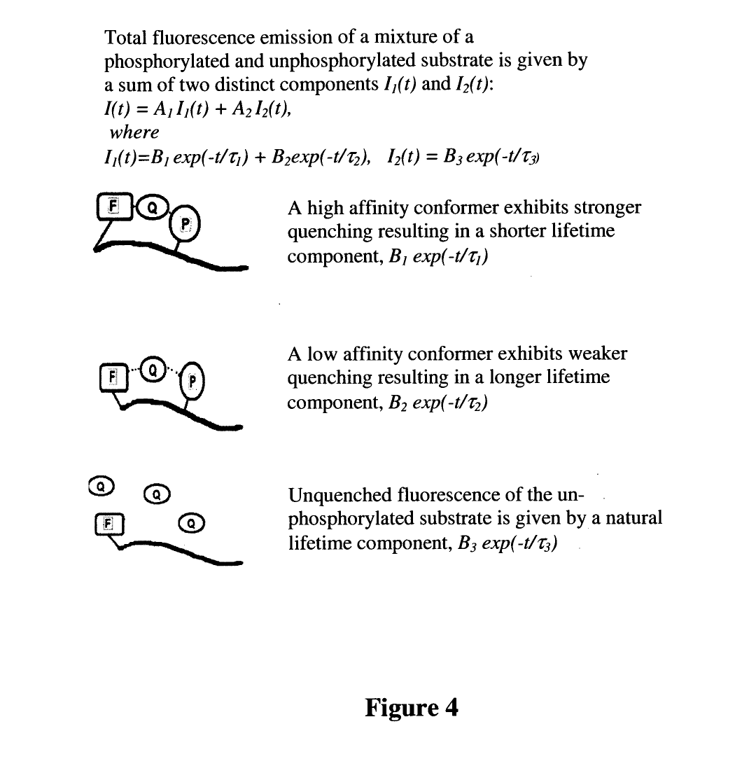

FIG. 4 shows an analysis of the interaction of the quencher with the AA9-labelled peptide substrate (crosstide peptide) of Protein Kinase B (PKB). This shows AA9 emission time-courses as a function of increasing concentrations of the quencher (left panel). The right panel shows analysis of these by a 3-exponential model based on:

$$I ( t ) = {\sum }_{i = 1}^{3} {B}_{i} exp ( - t / {τ}_{i} )$$

Amplitudes of the vertical lines represent normalized Bi coefficients. Positions of these lines on the X-axis correspond to the lifetimes of the respective exponential terms. All of these time-courses satisfactorily fit the 3-exponential model, and thus their lifetime pattern (spectrum) is characterised by three discrete components (δ-functions) in τ-space. The longest lifetime (16.38 ns) corresponds to emission of unquenched AA9 fluorophore, whereas the two shorter lifetime components (0.815 ns and 5.145 ns) correspond to the quenched emission of AA9. Remarkably, at a low quencher concentration (0.64 mM) the shortest lifetime component dominates in the AA2 emission. Increase in the quencher concentration to 1.25-2.5 mM gives rise to the second (intermediate) lifetime component (5.145 ns). A complete quenching of the long lifetime component is achieved at 2.5 mM quencher concentration.

The shortest lifetime component in AA9 emission (FIG. 3) appears at lower quencher concentrations. Its proportion however decreases along with increases in quencher concentrations and an additional component with intermediate lifetime appears. This confirms that the phosphorylated peptide forms two different affinity complexes with higher and lower quenching efficacy. A model (FIG. 4) for fitting emission time-courses of a mixture of the phosphorylated/unphosphorylated peptides is:

I(t)=A₁I₁(t)+A₂I₂(t),

where I₁(t)=B₁ exp(−t/τ₁)+B₂ exp(−t/τ₂) relates to emission of the phosphorylated substrate and I₂(t)=B₃ exp(−t/τ₃) to that of the unphosphorylated substrate. Therefore A₁ coefficient is proportional to concentration of the phosphorylated substrate whereas A₂ is proportional to concentration of the unphosphorylated substrate. The ratio of B₁ and B₂ coefficients in the I₁(t) function reflects the proportion of the higher and lower affinity complexes between the phosphorylated peptide and the quencher. This ratio as well as the lifetimes τ₁, τ₂ and τ₃ remain constant at a given quencher concentration. These model parameters can be determined with great accuracy in experiments where only phosphorylated or unphosphorylated peptides are present. Then evaluation of emission time-course of phosphorylated/unphosphorylated peptide mixtures is reduced to determination of the A₁ and A₂ coefficients. The latter are linear parameters and can be determined from data evaluation. Alternatively, time-courses of different samples can be evaluated together by the same model such that the respective τ-parameters have the same values for each time-course—global evaluation with linked τ-parameters. This approach as well as the evaluation with fixed at predetermined in control experiment values τ-parameters can significantly increase accuracy of determination of phosphorylated and unphosphorylated peptide concentrations.

Using the above model, the fraction of un-phosphorylated substrate, F₂, is given by:

F₂=A₂/(A₁+A₂).

The fraction of phosphorylated substrate, F₁, is given by:

F₁=A₁/(A₁+A₂).

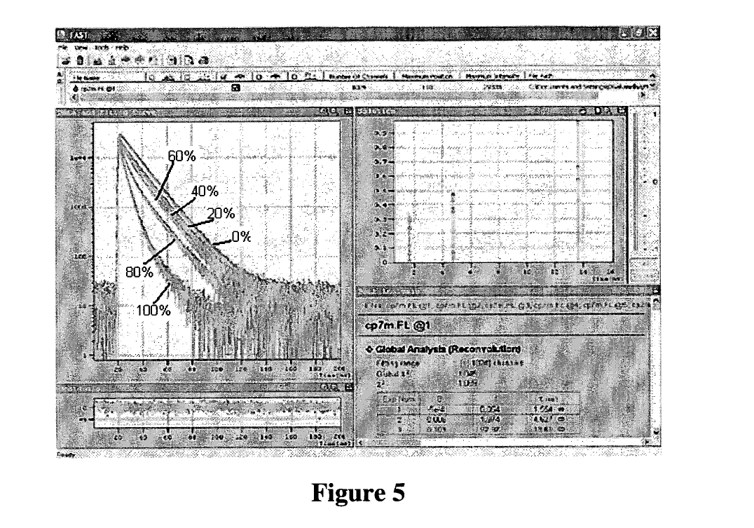

An example of the global evaluation of a family of fluorescence time-courses of mixtures of ACE14-labelled phosphorylated and unphosphorylated substrates containing gradually decreasing from 100 to 0 proportion of the phosphorylated peptide in the presence of 2.5 mM PMA/Fe³⁺ quencher is shown in FIG. 5. Each experiment was repeated 5 times to demonstrate the reproducibility and determine dispersion of the experimental parameters. The data were evaluated by using the above model with global linkage of the τᵢ-parameters. A remarkably good global value of the fit-quality parameter (X²=1.089) indicates a perfect correspondence of these experimental data to the 3-exponential model.

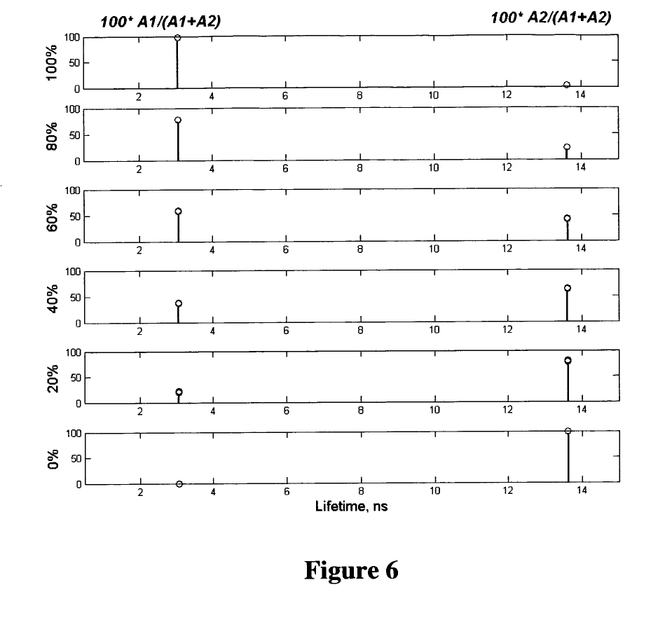

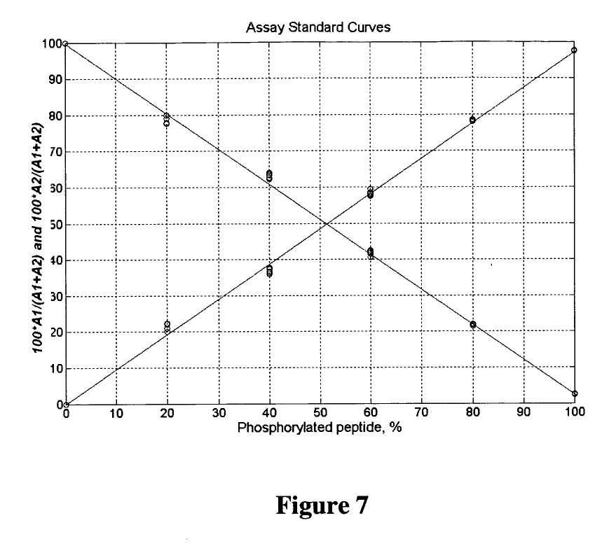

Normalized amplitudes of the quenched and unquenched components, (A₁)/(A₁+A₂)*100% and A₂/(A₁+A₂)*100%, for the different proportions peptide mixtures are shown in FIG. 6 by vertical lines of respective lengths. This shows that the quenched component amplitude is equal to 100% for 100% phosphorylated substrate and 0% for 100% unphosphorylated one. These amplitudes exhibit linear dependence as it is shown in FIG. 7. The Z-factor of this assay for different levels of substrate phosphorylation was calculated. The highest Z-factor exhibited (0.99) was for 100% phosphorylation. For the important phosphorylation range of 20-40% phosphorylation the Z-factor varies between 0.75-0.9, which is excellent.

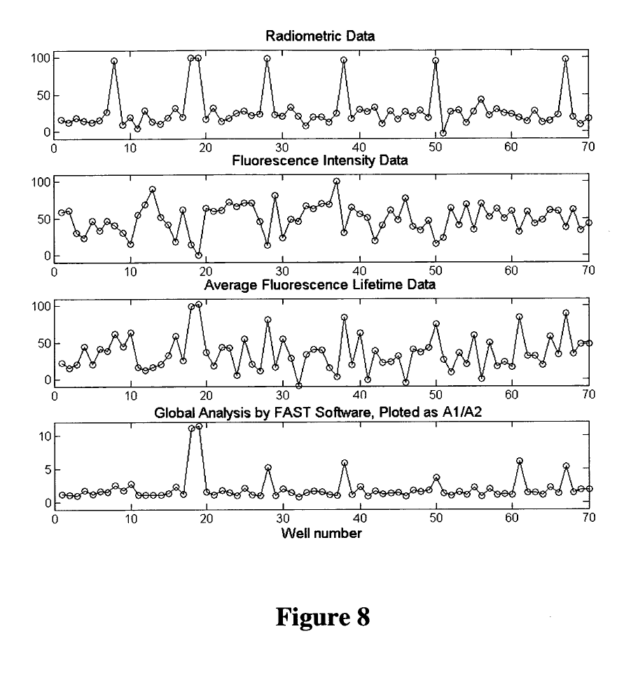

FIG. 8 illustrates further an advantage of using the model-based approach for data evaluation. The method allows increasing the “contrast” of the readout pattern by using the ratio of A₁/A₂. The latter parameter allows a five fold better discrimination of “hits” than the analysis based on the average lifetime.

The fluorescently labelled and test compounds subjected to the methods of the present invention may be based upon natural proteins, (including post-translationally modified proteins such as glycoproteins and lipoproteins), synthetic peptides, or their phosphorylated counterparts. Alternatively, the compounds may be based upon lipids, such as inositol lipids including phosphatidyl mono- or di-phosphates, or mono- or polysaccharides. By based upon is meant that the compounds comprise a protein or a peptide, for example, or a fragment thereof, in addition to any fluorescent label or phosphate moiety.

The compounds subjected to the methods of the present invention are typically peptides or proteins, in particular proteins, and the subsequent discussion focuses on these compounds although the invention should not be considered to be so limited.

Peptides are molecules made up of a plurality of amino acids joined together through their amino and carboxylic acid functionalities. The precise definition of what exactly is a peptide is invariably arbitrary in art; for example, a peptide may, in theory, comprise as few as 2 amino acids joined together (‘a dipeptide’). However, a peptide may be regarded more typically as comprising between 10 and 50 amino acids, more typically between 20 and 40 amino acids. In contradistinction, although the precise definition is again arbitrary, a protein may be regarded as a polymer of amino acids, that is to say, a molecule comprising very many amino acids and thus, for example, comprising more than 50 amino acids and more typically made from hundreds (e.g. up to 500) or even more amino acids. As this will be appreciated from the foregoing, however, the distinction between peptides and proteins is generally not the subject of a clear definition in the art. For the purposes of this invention, the point made here is merely that there is a distinction between peptides and protein recognised by those in the art. Moreover, as is known by those skilled in the art, some enzymes, for example kinases and phosphatases, may process peptides, or phosphopeptides (as appropriate), as their substrate whereas others, known as protein kinases or protein phosphatases, cannot process peptidic substrate but can only act upon proteins or phosphorylated proteins.

Typically the compounds will be peptidic, generally relatively short peptides comprising between about 5 and 30 amino acids.

In those aspects of the invention involving contact with a kinase, the test compound comprises at least one moiety susceptible to phosphorylation, generally an amino acid selected from tyrosine, serine, threonine and histidine, more particularly, serine or threonine. In these embodiments, the test compound is susceptible to phosphorylation by a protein kinase, for example a tyrosine, serine/threonine or histidine kinase, more particularly a serine/threonine kinase.

In these aspects of the invention involving contact with a phosphatase, the test compound comprises at least one phosphate moiety susceptible to dephosphorylation, generally a phosphorylated amino acid selected from tyrosine, serine, threonine and histidine, more particularly serine or threonine.

The nature of the test compound contacted with or exposed to the kinase or phosphatase enzymes according to the methods of this invention will generally depend upon the reason for conducting the method. For example, the method practised may serve as an assay to determine or to identify a phosphatase or kinase. In such cases, the purpose of the method may be to assay for the existence or identity of a kinase or phosphatase and the test compound may be a known, in some cases, generic, substrate, which may be defined as a substrate acted upon by more than one enzyme. Peptides known to be processed by more than one kinase or phosphatase, may be readily identified or known to those skilled in the art. For example, appropriate generic peptides in this regard are disclosed in WO03/087400. Alternatively, such peptides may be commercially available or known in the literature. An example of the lattermost is the synthetic peptide known as ‘Crosstide’ GRPRTSSFAEG D. A. (SEQ I.D. No. 1) (Cross, D. R. Alessi, P. Cohen, M. Andjelkovich and B. A. Hemmings “Inhibition of glycogen synthase kinase-3 by insulin mediated by protein kinase B” Nature 378(6559), 785-9 (1995)), a substrate PKB alpha, SKG 1 and other kinases. Other peptides may be purchased (e.g. by custom synthesis) as required.

On the other hand, the kinase or phosphatase used according to the invention may be known, in which case the reason for conducting the method or methods of the invention may be to identify a new substrate for a phosphatase or a kinase or to determine information, for example, usefulness of the test compound as a substrate or as an inhibitor. More details are provided hereinafter.

Where the activity of a kinase is to be investigated, the choice of the test compound with which to contact it will depend upon the particular kinase to be assayed. As known by the skilled person, substrate specificity of protein kinases varies considerably and it is known that the sequence adjacent to the phosphorylation site in the substrate plays an important role in its recognition by protein kinases. Thus, the selection of a particular test compound for a kinase assay will depend on the phosphorylation site motifs present in the sequence.

Increased knowledge of kinase substrate specificity has made it possible to identify potential enzyme recognition sites in newly sequenced proteins, and to construct synthetic peptide model substrates. For reviews in this field, see P. J. Kennelly and E. G Krebs, J. Biol. Chem., 266, 15555-58 (1991); B. E. Kemp and R. B. Pearson, Trends in Biochemical Sciences, 343 (1990). Likewise, suitable fluorescently labelled synthetic peptides may be prepared by methods that are well known to the skilled person, for example by solid phase synthesis involving the sequential addition of protected amino acids linked (optionally through a linker group) to a solid phase support, as described in “Solid Phase Peptide Synthesis”, E. Atherton and R. C. Sheppard, IRL Press (1989).

In vivo, the phosphatase catalytic domain is associated with and targeted by a regulator subunit. This means that phosphatase substrate specificity may be very specific in vivo. In vitro, however, phosphatases generally display broad substrate specificity, see N. R. Helps et al., Biochem. J., 349, 509-518 (2000); R. Majeti and A. Weiss, Chem. Rev., 101, 2441-2448 (2001); P. Cohen, J. Cell Science, 115, 241-256 (2002). Consequently, phosphatases in vitro are able to act on a wide range of both peptide and protein substrates.

Appropriate fluorescent labels may be acridones or quinacridones described respectively in WO02/099424 and WO02/099432. One such label known as ACE 14 as described in, for example, Example 1 of United States Patent Application Publication No. US2003/0228646 (ACE 14 is O-(N-Succinimidyl)-6-(9-oxo-9H-acrodin-10yl) hexanoate. Alternatively the label may be based upon an acridine derivative, for example of the type disclosed in WO2007/049057.

Labels particularly suitable for use in the present invention are fluorescence lifetime labels, the term “lifetime label” meaning a label having a measurable fluorescence lifetime, which is defined as the average amount of time that the label remains in its excited state following excitation (J. R. Lackowicz, Principles of Fluorescence Spectroscopy, Kluwer, Academic/Plenum Publishers, New York, (1999)).

Preferably the fluorescence label has a fluorescent lifetime in the range 5 to 30 ns, generally 8 to 25 ns, for example 12 to 25 ns.

Preferably, the label does not interfere with the activity (if any) of the enzyme on the test compound. However, it will be appreciated that this is not an absolute requirement in, for example, the assaying of possible or putative substrates against a given enzyme in high throughput screening applications: what is typically required in such assays is the identification of ‘hits’ and thus the identification of qualitative, as opposed to quantitative, leads.

As discussed in greater detail hereinbelow, the invention contemplates in particular the use of peptides and proteins in which the fluorescent label, where present in the test compound, may be attached to a terminal amino acid or to one or more (normally one) internal amino acids.

The fluorescently labelled compounds may be made using chemistry well known to the skilled person. For example amine-reactive or thiol-reactive fluorescent labels may be used. Examples of amine-reactive fluorescent labels are isothiocyanato- and N-hydroxysuccinimidyl ester derivatives of fluorescent labels; these may be reacted with the amino sidechain of lysine or the amino-terminus in peptides or proteins. Whilst the use of thiol-reactive fluorescent labels is less common, since the thiol groups of amino acids are generally present in disulfide crosslinkages, thiol labelling is possible, for example using iodoacetyl and maleimidyl derivatives of fluorescent labels. For reviews and examples of protein and peptide labelling using fluorescent labelling reagents, see “Non-Radioactive Labelling, a Practical Introduction”, A. J. Garman, Academic Press (1997); “Bioconjugation-Protein Coupling Techniques for the Biomedical Sciences”, M. Aslam and A. Dent, Macmillan Reference Ltd (1998). Protocols are available to obtain site specific labelling in a synthesised peptide, for example, see G. T. Hermanson, Bioconjugate Techniques, Academic Press (1996).

In the fluorescently labelled compounds used in the methods of this invention there is a relationship between the fluorescence, and so the fluorescence lifetime, and the distance between the position of (i) the phosphate group in the compound or the position of the moiety susceptible to phosphorylation and (ii) the fluorescent label. Where the phosphate or moiety susceptible to phosphorylation is closer to the fluorescent moiety, the distinction between fluorescence and fluorescence lifetime of the compounds and their resultant product, if processed by the enzyme, are greater.

Where the fluorescently labelled compound is the test compound, and is a peptide or a protein, therefore, it may be advantageous to site the phosphate or moiety susceptible to phosphorylation on or as an amino acid residue adjacent to that to which the fluorescent label is attached. It will sometimes be the case, however, that this is not advantageous since more efficient processing by the enzyme may be achievable if the phosphate or moiety susceptible to phosphorylation is present at or as a non-adjacent amino acid. There is an inverse relationship with distance and so, preferably, the phosphate or moiety susceptible to phosphorylation is present at or as an amino acid 10 or fewer positions away from the amino acid to which the fluorescent label is attached, more preferably fewer than six amino acids away, still more preferably fewer than three amino acids away. In this nomenclature one amino away refers to the phosphate or moiety susceptible to phosphorylation being on or as the amino acid adjacent to that to which the fluorescent label is attached. Generally the phosphate or moiety susceptible to phosphorylation will not be present at or as the same amino acid to which the fluorescent label is attached although it will be understood that this is possible since the fluorescent label can be attached to the C or N terminus. Generally, however, the phosphate or moiety susceptible to phosphorylation will be present at or as an amino acid one or more away from that to which the fluorescent label is attached.

In a particular embodiment of this invention, the fluorescent label will be attached to a terminal amino acid, generally via the C or N terminus, typically the N terminus.

Where the activity of a phosphatase is to be determined according to the fifth and eighth aspects of this invention, the test compound comprises a phosphate group. Where the compound is based upon a peptide, or a protein, the peptide or protein may be synthesised prior to it being phosphorylated. Alternatively, one or more phosphorylated amino acids may be incorporated into the compound during synthesis. Appropriate phosphorylated amino acids are commercially available, for example from Bachem AG.

Labelling of a phosphorylated compound with the fluorescent label may be carried out as described above during the synthesis of the peptide, either by the use of a labelled amino acid in the synthetic process, or by the specific deprotection and labelling of the residue of interest before deprotection of other potentially reactive residues at the completion of the synthesis. Protein phosphatase substrates are in, or converted into, a suitable phosphorylated form for use in the assays according to the invention. The phosphorylated compound may then be labelled, if appropriate, with a suitable fluorescent label.

In some embodiments of the invention, the test compounds contacted with the kinase or phosphatase may be bound to a solid support by a linker group. This enables the methods of the invention to be practised in the solid phase, which lends itself to high throughput applications. More generally, the methods of the present invention may be used in various assays applicable to life sciences, biotechnology and drug discovery, to provide quantitative information on the activity of kinases and phosphatises or the ability of a compound of interest to inhibit (or promote) enzymatic activity. The homogeneous sample formats allows straightforward application in high throughput screening.

In other embodiments of the invention, the test compounds contacted with the kinase or phosphatase may be attached to a further compound, for example a transport peptide as described in U.S. Pat. No. 5,807,746; WO99/64455; WO97/12912; WO99/05302, Rojas et al., Nature Biotechnology, 16, 370-375 (1998); Hawiger et al., Curr. Opinion Chem. Biol., 89-94 (1999)). Alternatively the transport peptide may be Penetratin (Cyclacel, UK), for example TAT or Chariot. In such embodiments, the transport peptide can, for example, transport the test compounds across a cellular membrane and into a cell so as to enable the study of the test compounds in a cellular environment. When it is wished to deliver such a compound to cells grown in cell or tissue culture, the compound is simply added to the culture medium.

In these ways, the methods described herein are of utility in the investigation or kinase or phosphatase activities in crude cell lysates or, indeed, whole cells.

In contrast to the practice of the fourth and fifth aspects of the invention, the fluorescently labelled compound used in accordance with the seventh and eighth aspects of the invention is not the test compound and is intended to act as a substrate for the enzyme. Rather, the fluorescently labelled compounds employed in these aspects of the invention function as reporters that allow information to be obtained into an attendant (de)phosphorylation reaction it is wished to study.

In the seventh aspect of the invention, the kinase, and in the eighth aspect, a phosphatise, is exposed to the test compound in the presence of a complex comprising a fluorescently labelled phosphorylated compound and a protein. The presence of quenching moieties, for example the aromatic side chains in the amino acids tyrosine, tryptophan and phenylalanine in the protein, serves to quench the fluorescence from the fluorescently labelled phosphorylated compound when it is bound to the protein.

As and when the test compound is processed by the kinase, a phosphorylated compound is produced, the phosphate groups of which compete with the fluorescently labelled phosphorylated compounds for binding to the complex.

These phosphorylated (non-fluorescently labelled, phosphorylated test compounds) will displace a proportion of the fluorescently labelled phosphorylated compounds. This displacement serves to separate the fluorescent labels from the quenching moieties present in the protein and so causes an increase in fluorescence, which can be measured. It is in this way that the fluorescently labelled phosphorylated compounds function as reporters allowing information to be obtained into the extent of phosphorylation by the kinase.

As well as the protein, any molecule could be used that may be modified to allow interaction or coordination to the phosphate groups of both the fluorescently labelled phosphorylated compounds and phosphorylated substrates may bind.

The tertiary and quaternary, in particular tertiary, structures of proteins have the effect that, inevitably, one or more suitable quenching amino acids will be present at (a) close enough position(s) to a fluorescently labelled phosphorylated compound, when bound, to effect quenching of the fluorescent label. Additionally, proteins may be modified so as to introduce additional quenching moieties such as those disclosed herein. It is for this reason that the choice of protein is not particularly limited. The protein may be any convenient protein. Examples include bovine serum albumin (BSA), histones and myelin basic protein (MBP).

In certain embodiments the protein may be modified to attach one or more moieties that serve to interact with, or coordinate to, a phosphate group. An example of such a moiety is the iron (III) ion. Typically, a plurality of moieties, e.g. iron (III) ions, is attached. With BSA, for example, we calculate that about 20 iron (III) ions per BSA molecule may be attached.

Typically, the protein is modified by attaching one or more co-ordinating, or chelating, entities thereto, which is or are capable of binding the moieties which serve to interact with, or coordinate to, phosphate groups, e.g. iron (III) ions. We find diethylenetriamine pentaacetic acid to be an appropriate entity; others will be known to those skilled in the art. The entity may be introduced to the protein by attachment to an appropriate amino acid side-chain. For example reaction of diethylenetriamine pentaacetic acid anhydride may be incubated with BSA to effect diethylenetriamine pentaacetic acid labelling by way of reaction of the anhydride with the side chain amino groups of lysine residues. It will be appreciated that it is possible to vary the extent of moieties, e.g. iron (III) ions, introduced by variation in the extent of chelate moiety introduced. This in turn may be controlled either by varying the proportion of chelating group with which the protein is incubated, and/or by selecting a protein with a higher or lower proportion of labelling sites. Such modifications are within the ability of a person skilled in the art.

Once the chelating or co-ordinating entities have been introduced, the moieties that serve to interact with or coordinate to phosphate groups, e.g. iron (III) ions, are attached to the resultant protein in any convenient way. We find exposure to iron (III) perchlorate in aqueous (e.g. 50% v/v) acetic acid to be an appropriate way in which to introduce Fe (III) onto BSA. Unreacted or excess iron (III) may be easily removed by gel filtration.

In order to make the complexes proteins capable of binding to phosphate groups (including those described hereinbefore) are exposed to the fluorescently labelled phosphorylated compound. Unlike the other aspects of the invention discussed hereinbefore, the nature of the fluorescently labelled phosphorylated compound need not be dictated (in any way) by it serving as a substrate or inhibitor, of the kinase. Accordingly any compound may be used. We find modified peptides to be convenient in this regard, for example fluorescently labelled and phosphorylated Crosstide peptide is suitable.

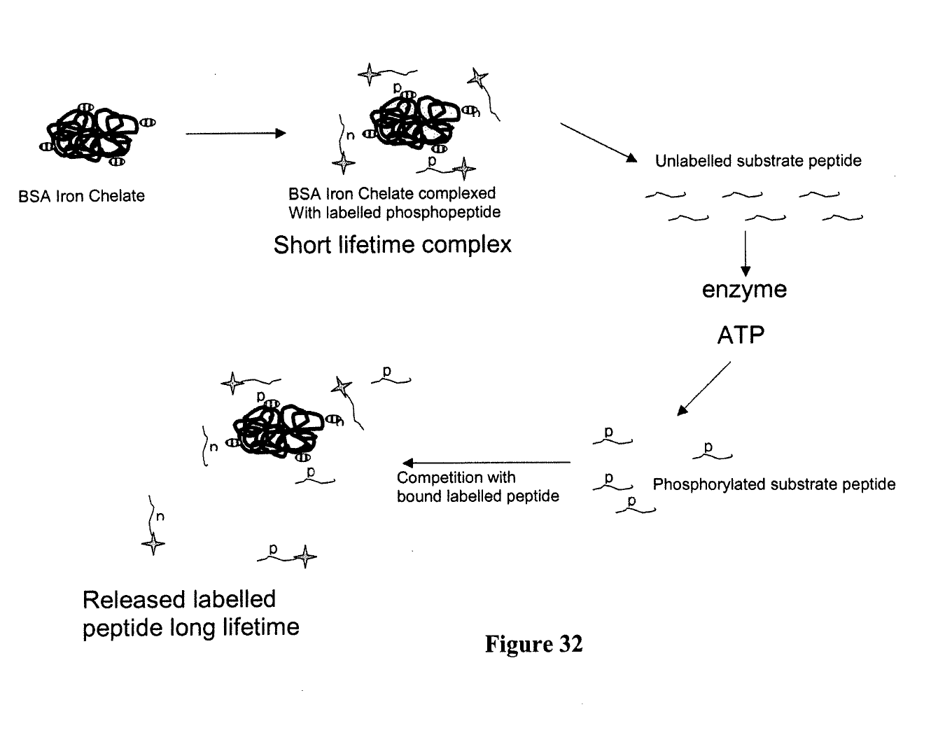

In the practice of the seventh aspect of this invention a kinase is contacted with a substrate in the presence of a complex as described herein. An example of this is shown schematically in FIG. 32 in which the weight ligand complex is based upon BSA.

The fluorescence parameter measured according to the practice is the method of this invention may be any method of quantifying fluorescence, for example fluorescence intensity or fluorescence lifetime. Additionally practice of the seventh and eighth aspects of the invention may involve use of the well-known techniques of fluorescence polarization and fluorescence lifetime polarization. As is known in the art the latter two techniques may be employed since the fluorophore labelled phosphopeptide is present initially in a complex with a protein (having high polarization) prior to its dissociation, caused by completion with phosphorylated non-labelled compound, typically a peptide. The free labelled peptide has significantly lower molecular mass resulting in faster fluorescence depolarization than that of its protein-bound counterpart.

When practice of the seventh and eighth aspects of the invention involves fluorescence polarization and fluorescence lifetime polarization, quenching of fluorescence from the fluorescent label is not required because the bound or unbound state of fluorescently labelled compound can be distinguished solely by virtue of the polarization aspect to the parameter measured.

In one embodiment of the invention, the fluorescence parameter measured during the contacting step according to the methods of this invention is measurement of fluorescence lifetime. If the test compound is acted upon by the kinase or phosphatase, a change—either an increase or a decrease—in fluorescence and in fluorescence lifetime is detectable. Where the fluorescently labelled phosphorylated compound is released from the protein complex, according to the seventh or eighth aspects of the invention, a decrease in fluorescence polarisation, increase in fluorescence lifetime and increase in fluorescence intensity are detectable.

Conventional detection methods can be employed to measure fluorescence intensity and/or the lifetime of the fluorescent label and/or fluorescence polarization (and/or fluorescence lifetime polarization). Such methods make use instruments using photo-multiplier tubes as detection devices. Several approaches are possible using these methods, such as:

i) methods based upon time-correlated single photon counting (cf. Principles of Fluorescence Spectroscopy, (Chapter 4) ed. J. R. Lakowicz, Second Edition, Kluwer/Academic Press (1999);

ii) methods based upon frequency domain/phase modulation (cf. Principles of Fluorescence Spectroscopy, infra, Chapter 5; and

iii) methods based upon time-gating (cf. Sanders et al., Analytical Biochemistry, 227(2), 302-308 (1995)).

Suitable devices are the Edinburgh Instruments FLS920 and FL900CDT spectrometers (Edinburgh Instruments, UK) or Fluorescence Lifetime Plate Reader the NanoTourus employing time-correlated single photon counting method which provides the instruments with high sensitivity and ca 100 ps temporal resolution. A new Fluorescence Analysis Software Technology (FAST) used in these instruments allows reliable and accurate the evaluation of experimental data.

Measurement of fluorescent intensity may be performed by means of a charge coupled device (CCD) imager, such as a scanning imager or an area imager, to image all of the wells of a multiwell plate. The LEADseeker™ system features a CCD camera allowing imaging of high density microtitre plates in a single pass. Imaging is quantitative and rapid, and instrumentation suitable for imaging applications can now simultaneously image the whole of a multiwell plate, e.g. a microtitre plate having 24, 96,384 or higher densities of wells, e.g. 1536 wells.

A particular advantage of determining fluorescence lifetimes, in addition to these discussed above, arises from the ability to distinguish one fluorophore from another if it has a different lifetime. Thus it is possible to practice the methods of this invention in the presence if two or more fluorescently labelled compounds where the fluorophores have different lifetimes. This may be useful in simultaneous investigations of an enzyme's activity against two such compounds.

In practising the methods of the invention, the compounds (i.e. the putative or potential substrates) for the phosphatase or kinase being used is contacted with that kinase or phosphatase and the fluorescence measured during the contacting. In addition to the presence of the kinase or phosphatase and the fluorescently labelled compound, it will be appreciated that there will also be present other components sufficient to form an environment which will allow phosphorylation or dephosphorylation to occur where the fluorescently labelled compound is an appropriate substrate for the kinase or phosphatase. Thus, for example, the contacting will typically be formed in an aqueous medium and where the enzyme contacted is a kinase, that source of phosphate, such as ATP (generally ATP) or GTP will be present. Typically, the aqueous medium will be buffered (e.g. with Tris, HEPES or MOPS, in particular MOPS), typically with a source of magnesium (e.g. MgCl₂) and at a pH of about 7 to 9. Such are merely standard conditions for the functioning of kinase and phosphatase enzymes and will be known to those skilled in the art. An example of a suitable medium is one comprising 0.5 mM ATP, 5 mM MgCl₂ and 50 mM Tris at pH7.

Typically, kinase assays are performed under “stopped” conditions. Thus, the reaction is allowed to proceed for a predetermined time and then the reaction is terminated with a stop reagent, normally an inhibitor of the enzyme activity, which is often non-specific. An example of a stop reagent is EDTA, which is used to sequester metal ions such as Mg²⁺ that are normally required for kinase activity. Alternatively, and as discussed below, the stopped conditions may be provided by addition of acid (an “acidic stop”). Conveniently the quenching moieties may be provided in an acidic solution.

The methods of the present invention are typically conducted in the presence of a moiety that causes a change in the fluorescence of the probe. This can either be by enhancing or increasing the fluorescence intensity or fluorescence lifetime or by quenching the fluorescence intensity or fluorescence lifetime. By quenching is meant herein that the quenching moiety serves to reduce the fluorescence intensity, or fluorescence lifetime, of the fluorophore as compared with the fluorescence intensity, or fluorescence lifetime, in the absence of the quenching moiety.

An example of a particular class of compounds that provides the quenching moiety is iron (III) chelates, that is to say complexes formed between the iron (III) ion and one or more polydentate ligands that serve as the quenching moiety.

In certain embodiments of the seventh and eighth aspects of the invention iron (III) chelates are provided as part of the protein-containing complex, as discussed hereinbefore.

In the fourth and fifth aspects of the invention the quenching moiety is typically provided by an aromatic- or heteroaromatic-containing polydentate ligand, typically aromatic-containing bi- or tridentate ligands. Typically such moieties are provided within chelates such as those formed with Fe(III). Examples include chelates formed between Fe(III) and phenylmalonic acid and between Fe(III) and 2-hydroxyacetophenone.

In one particular embodiment according to the fourth and fifth aspects of the invention, the iron (III) chelate is a complex formed by contacting an iron (III) salt in a 1:1 molar ratio with an aromatic- or heteroaromatic-containing polydentate ligand. We find a particularly convenient reagent to be prepared by mixing approximately equimolar quantities of iron (III) with phenylmalonic acid in aqueous acid, for example aqueous acetic acid. Use of this reagent is advantageous in that addition of the reagent also serves as a stop reagent, since the presence of the acid denatures the enzyme.

In one embodiment, the methods of the invention may be used to assess the phosphorylating action of a kinase, or dephosphorylating action of a phosphatase, in the presence of an appropriate fluorescently labelled compound. In some aspects the test compound will be capable of being processed by the enzyme and so, by monitoring the fluorescence of the compound and the resultant product produced from it by action of the enzyme, the activity of the enzyme can be determined.

In certain embodiments of this invention the methods may be conducted with a second test compound, in addition to the presence of the first test compound where, for example, the first test compound is known to be a substrate of the kinase or phosphatase. The second test compound may be, for example, an inhibitor or potential inhibitor of the enzyme. In this way the methods of the invention may be used to assay potential phosphatase or kinase inhibitors. It will be appreciated that more than one such second test compound may be present at a time, permitting the screening of libraries of compounds in drug discovery programmes, for example, to identify kinase or phosphatise inhibitors. Such or other methods of the invention may be conducted in the wells of a multiwell plate, in assay tubes or in the microchannels of a microfluidic device.

In another embodiment of the invention, the methods may be used to monitor the suitability of a test compound when contacted with a kinase or phosphatase, to determine its appropriateness as a substrate, or inhibitor, of the enzyme.

The invention is illustrated below by the following non-limiting examples.

### EXPERIMENTAL

### Materials and Methods

All chemicals were obtained from Sigma Aldrich and were “Analar” grade or better except where specifically noted.

Peptides were synthesised commercially by Bristol University. Three such peptides used (see Example 17) are:

| | | |
|---|---|---|
| Pep1 | RARTLSFAEPG | (SEQ I.D. No. 2) |
| Pep2 | RRRLSFAEPG | (SEQ I.D. No. 3) |
| Pep3 | KKLNRTLSFFAEPG | (SEQ I.D. No. 4) |

The peptides were labelled at the N-terminal by ACE14.

Phosphorylation occurs at the serine, which is 5, 4 or 7 amino acids from the fluorophore for Pep1, Pep2 and Pep3 respectively.

### Peptide Labelling.

Peptides were labelled with ACE-14 as the NHS ester as follows: lyophilised peptide (1 mg) was dissolved in 0.1 M sodium carbonate buffer at pH 8.0 and a 1.5 M excess of ACE-14 dye dissolved in DMSO was added. The reaction was incubated overnight at 4° C. and then quenched by addition of a 2 molar excess of ethanolamine and incubated at room temperature for a further 1 h. The dye-labelled peptide was then purified by HPLC on a Phenomenex C18 reverse-phase column using an acetonitrile gradient. The identity and purity of the peptide was confirmed by mass spectrometry.

### Radiometric PKB Assay.

The radiometric PKB assay was carried out as detailed (Cross, D. R. Alessi, P. Cohen, M. Andjelkovich and B. A. Hemmings “Inhibition of glycogen synthase kinase-3 by insulin mediated by protein kinase B” Nature, 378(6559), 785-9 (1995)).

### Iron III Chelates

The iron (III) chelates were prepared as follows. The chelate molecules (phenylmalonic acid (PMA) or 2-hydroxyacetophenone (HAP)) were dissolved in water to a concentration of 0.1 M. The iron (III) perchlorate was dissolved in 100% glacial acetic acid to a concentration of 0.1 M. The chelate complex was formed by mixing equal volumes of the required chelate and acidic iron (III) solution. The iron (III) chelate mix was then added to peptide samples or assays as required.

### Lifetime Assay for PKB and Other Test Enzymes

PKB and test enzymes were used at concentrations listed in figure legends with a substrate mix shown below.

Substrate mix 20 μM ATP, 2 mM MgCl₂ and 25 μM dye-labelled Crosstide or enzyme-specific peptide (final concentrations) added in 15 μl, Enzyme added in 10 μl. Assay stopped by addition of 25 μl of 2 mM chelate in 50% acetic acid.

### Screening Trial

For the screening trial (Experiment 16) 96-well plates were prepared containing 2 μl of inhibitors in DMSO per well with controls congaing DMSO alone (100% activity) and wells containing boiled enzyme (100% inhibition). Enzyme was added to wells in a volume of 10 μl and pre-incubated for 10 min before the addition of substrate mix as detailed above. The assays were terminated by the addition of iron (III) chelate in 50% acetic acid to a final concentration of 2 mM. Fluorescence lifetimes were then determined using the nanotaurus plate reader.

### Experiment 1

This experiment demonstrates that labelling of known substrate peptides did not take away their ability to act as substrates.

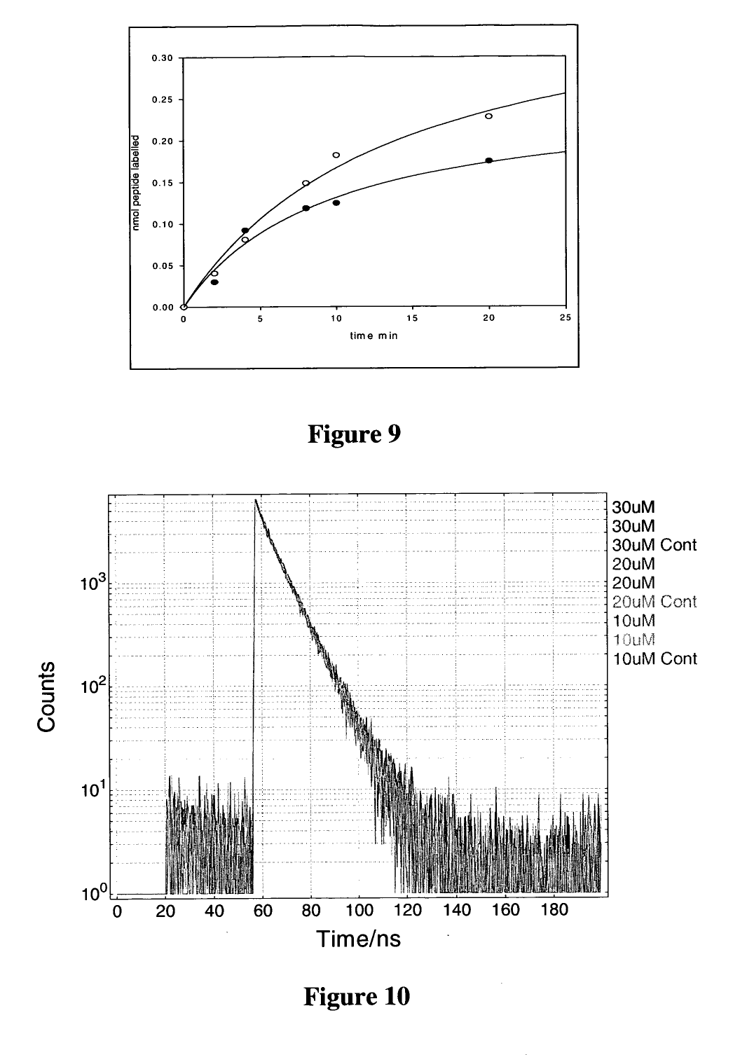

The Crosstide synthetic substrate for PKB alpha (GRPRTSSFAEG (SEQ I.D. No. 1)) was selectively labelled at the N-terminus using the succinimidyl ester of the propriety ACE 14 as described in Materials and Methods above. The results, shown in FIG. 9, show that although there is a reduction in the reaction rate using the labelled peptide compared to unlabelled it is still a viable substrate.

A radiometric PKB assay was conducted as described in Materials and Methods above. Samples were taken for determination of incorporation of ³³P at the time points indicated. The assays contained either unlabelled (∘) or labelled () peptide at a concentration of 30 μM.

### Experiment 2

This experiment (into the effects of phosphorylation on fluorescence lifetime) again made use of the synthetic Crosstide peptide. In order to gauge the effect of peptide phosphorylation on fluorescence lifetime three concentrations of labelled peptide (30, 20 and 10 μM) were assayed in duplicate under the same conditions as for the radiometric assay, except the concentration of ATP was increased to 0.5 mM. In the control samples the PKB was heated for 5 min at 70° C. prior to use. Assays were terminated after 30 min incubation at 25° C. by the addition of EDTA to a final concentration of 15 mM.

Fluorescence lifetime decay curves for PKB assays conducted in duplicate as described in Materials and Methods with labelled peptide at the concentrations shown. Controls (Cont) were assays conducted with boiled enzyme.

The results (shown in FIG. 10) clearly show no change in lifetime under conditions which gave phosphorylation of the peptide in the radiometric assay. Phosphorylation of the peptide was confirmed by mass spectrometry (data not shown)

### Experiment 3

This experiment shows that the distance of the phosphate from the label per se is not a factor in the determining the ability of the phosphate to affect the lifetime of the label.

A series of synthetic phosphorylated peptides were obtained with a lysine which can be labelled with the reporter label of choice at increasing distances from the phosphorylated residue as shown in Table 1.

TABLE 1
shows the series of N-acetylated synthetic
phosphopeptides labelled with ACE 14 label on
the C-terminal lysine. S(Pi) indicates the
position of the phosphoserine residue.

| | |
|---|---|
| Ac-GSPNANK | [1] |
| Ac-GS(Pi)PNANK | [2] |
| Ac-GPS(Pi)NANK | [3] |
| Ac-GPNS(Pi)ANK | [4] |
| Ac-GPNAS(Pi)NK | [5] |
| Ac-GPNANS(Pi)K | [6] |

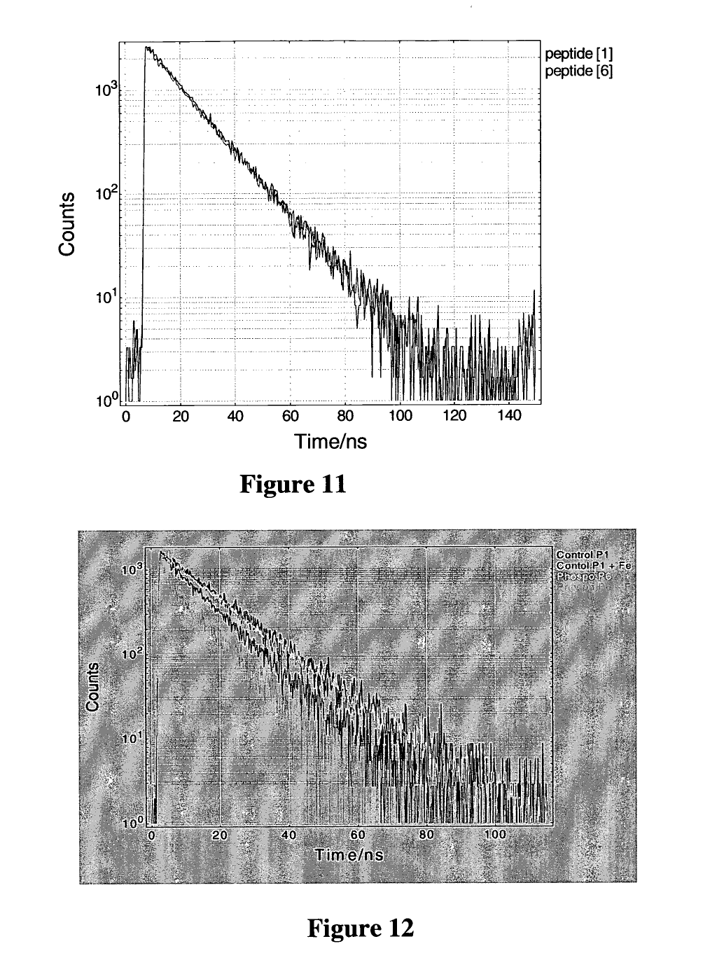

A comparison of the fluorescence lifetime of the control peptide [1], containing no phosphate, and the peptide with the phosphate adjacent to the labelled lysine [6] showed no difference in lifetime (see FIG. 11, which shows lifetime decay curves for unphosphorylated test peptide [1] and phosphopeptide [6] with the phosphorylated residue next to the reporter label). From this it is clear that the proximity of a phosphate per se is not sufficient to change the lifetime of the label.

### Experiment 4

Having established (Experiment 3) that the presence of a phosphate group alone is insufficient to alter the fluorescent lifetime, the effect of various ionic species was investigated. Of the salts tested, only iodide, nickel(II), copper(II) and iron salts had any significant effect on lifetime with iodide quenching the lifetime almost completely, in a concentration-dependant manner. Significantly, sodium phosphate also failed to affect the fluorescence lifetime (data not shown).

FIG. 12 shows that iron (II) as ferrous sulphate on its own results in a lifetime change in both the phosphorylated and unphosphorylated peptides in a concentration dependent manner. In particular, the figure shows that iron (II) as ferrous sulphate on its own results in a differential lifetime change in both the phosphorylated and unphosphorylated peptides in a concentration dependent manner, since the magnitude of the change between the phosphorylated and unphosphorylated peptides is different.

Use of the ferrocenyl derivative shown below:

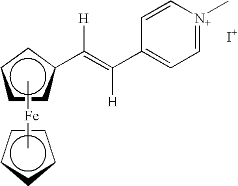

was found to be susceptible to interference by biological buffers.

### Experiment 5

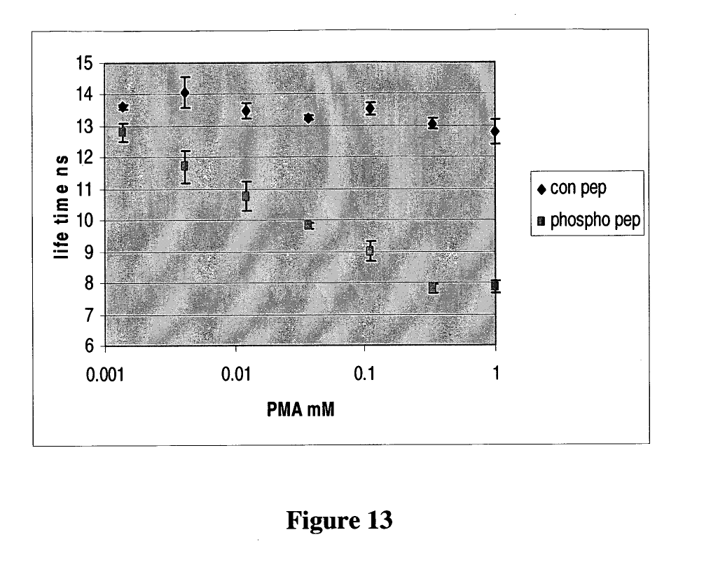

The chelate formed between iron(III) and phenylmalonic acid (PMA) was found to be effective in decreasing the lifetime of the label in a phosphate dependent manner as shown in FIG. 13. This shows a plot of lifetime fluorescence against increasing amounts of chelate for peptides 1 and 6 (“con pep” and “phospho pep” respectively) described in Experiment 3, at 5 μM.

### Experiment 6

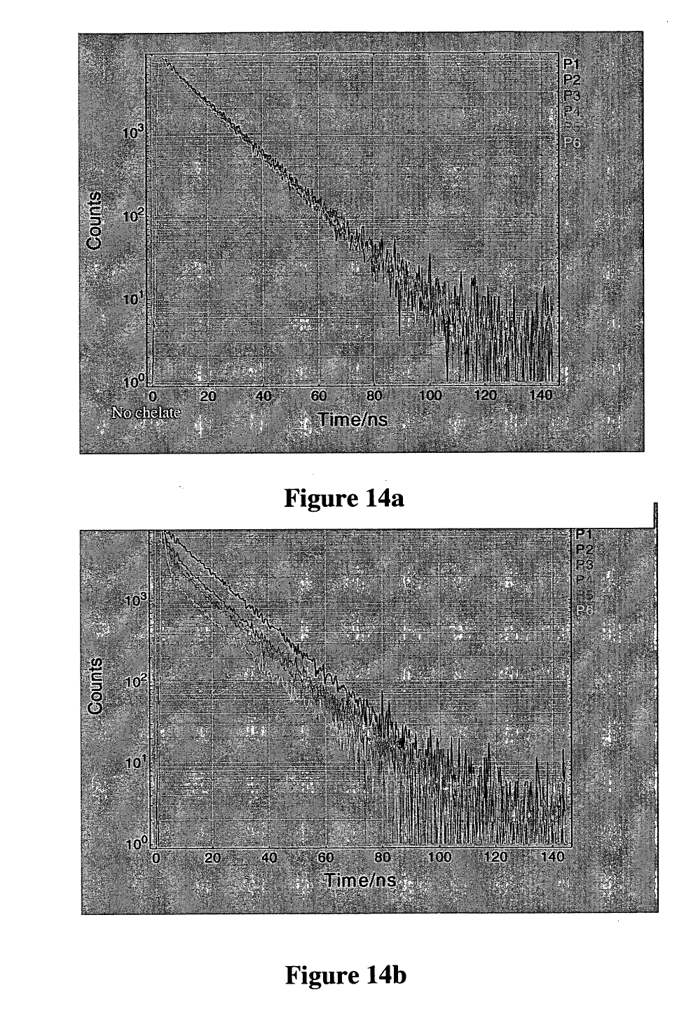

The distance over which the effect of the iron (III) PMA chelate could be observed was also investigated using the previously synthesised phosphopetide series [1]-[6] above (Experiment 3). The data presented in FIG. 14 in which P1 is peptide [1].

FIG. 14A shows the lifetime decay curves for the peptide series without iron chelate. FIG. 14B shows the decay curves for the peptides in the presence of the iron chelate.

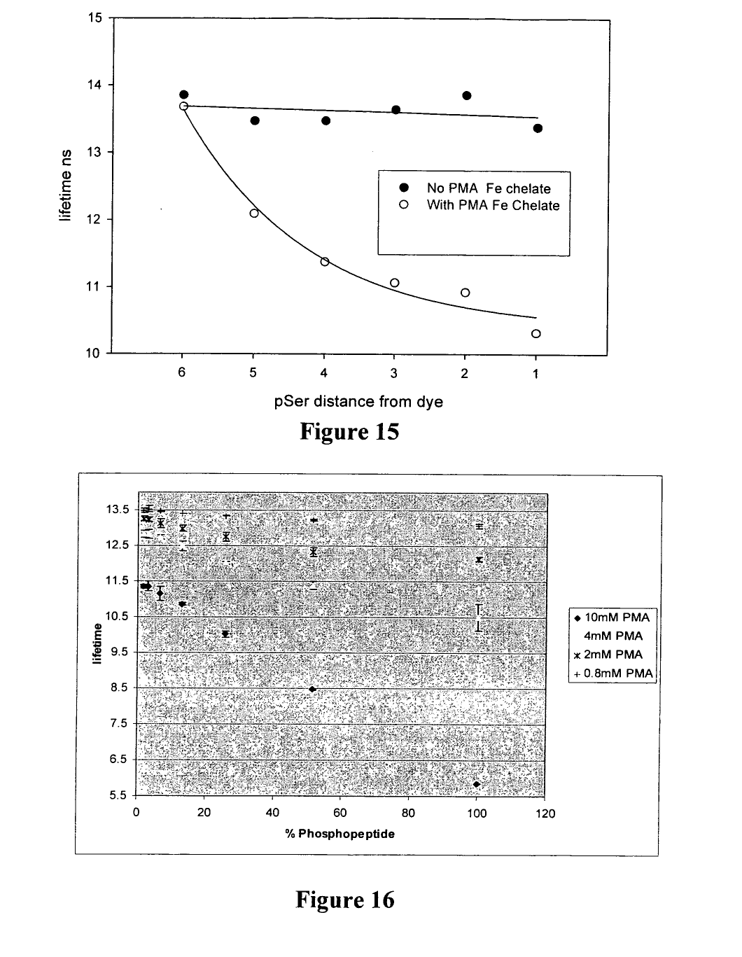

A plot of the lifetime against distance from the phosphorylated residue (FIG. 15) in the peptide shows a regular relationship. Note that, in FIG. 15, distance 6 corresponds to the control unphosphorylated peptide [1].

After further experiments to determine buffer compatibility and stability of the chelate it was determined that the optimum buffer system was MOPS and the chelate was most stable as a 1:1 Fe(III):PMA in 50% acetic acid.

### Experiment 7

To determine the sensitivity of the assay for phosphopeptide a series of peptide mixtures containing the control unphosphorylated peptide [1] and in increasing mole fractions of phosphopeptide [6] were prepared and the fluorescence lifetimes measured with different concentrations of the PMA Fe(III) chelate.

The results in FIG. 16 show that a 10 to 20% substrate conversion is easily detectable and therefore the detection sensitivity is in the range required for initial rate measurements of enzyme activity.

### Experiment 8

Using the above methodology a PKB assay was developed using a substrate mix comprising ATP, MgCl₂ and labelled Crosstide in a volume of 15 μl which was added to 10 μl of recombinant PKB enzyme giving final concentrations of 20 μM ATP, 2 mM MgCl₂ and 20 μM Peptide. After incubation at 25° C. for 40 minutes the assay was stopped by addition of 25 μl of 2 mM iron chelate in 50% acetic acid.

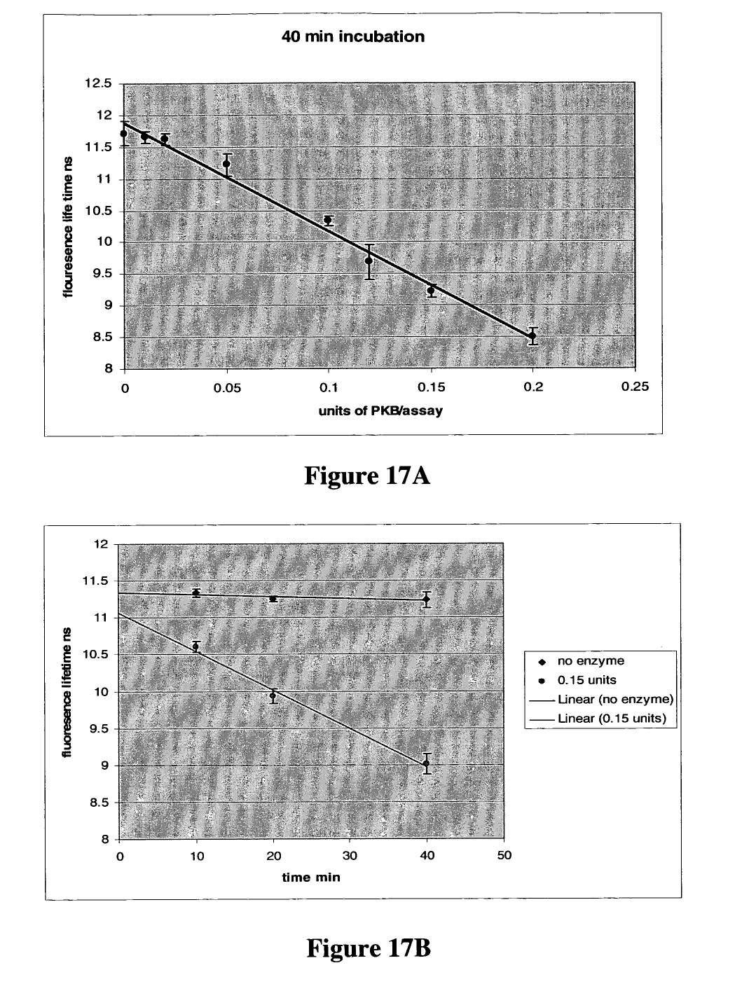

The data shown in FIG. 17A shows a linear dependency on protein concentration and, using the same reaction conditions, a time course of the assay is shown in FIG. 17B.

### Experiment 9

In order to be of use in screening an assay must be reproducible. The standard measure or reproducibility and signal to noise discrimination is the Z′ Factor. This is obtained by statistical analysis of multiple sets of assays, in this case seven groups of 4 experimental and four control data points. In this case the controls were carried out using boiled PKB. All other parameters were as for the assay described in Experiment 8.

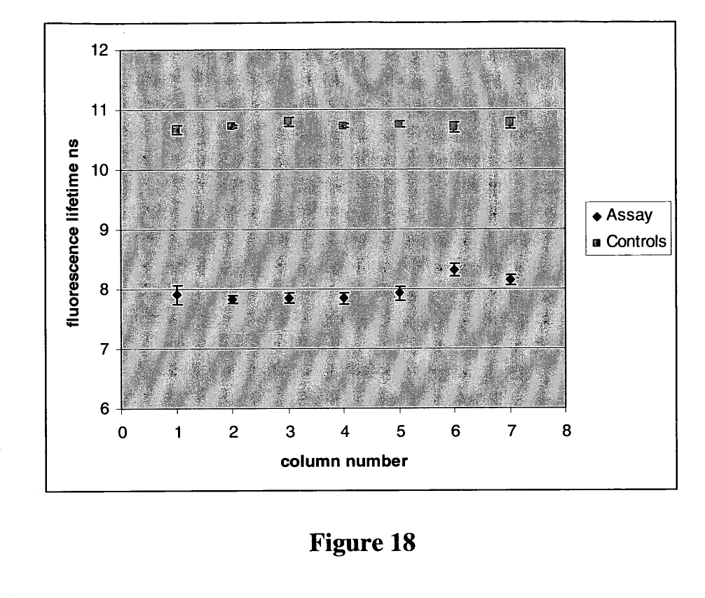

Each data point presented in FIG. 18 is the mean and SD of 4 replicates, from this data the calculated Z′ factor for the assay is 0.75 indicating that this assay is viable for use in high throughput screening.

### Experiment 10

The assay was also trialled with two other enzymes MAPKAP K2 and SGK1. The assay for MAPKAP K2 was essentially as described above for PKB with the exception that the substrate peptide was N-acetylated KLNRTLSVA with the label on the lysine side chain.

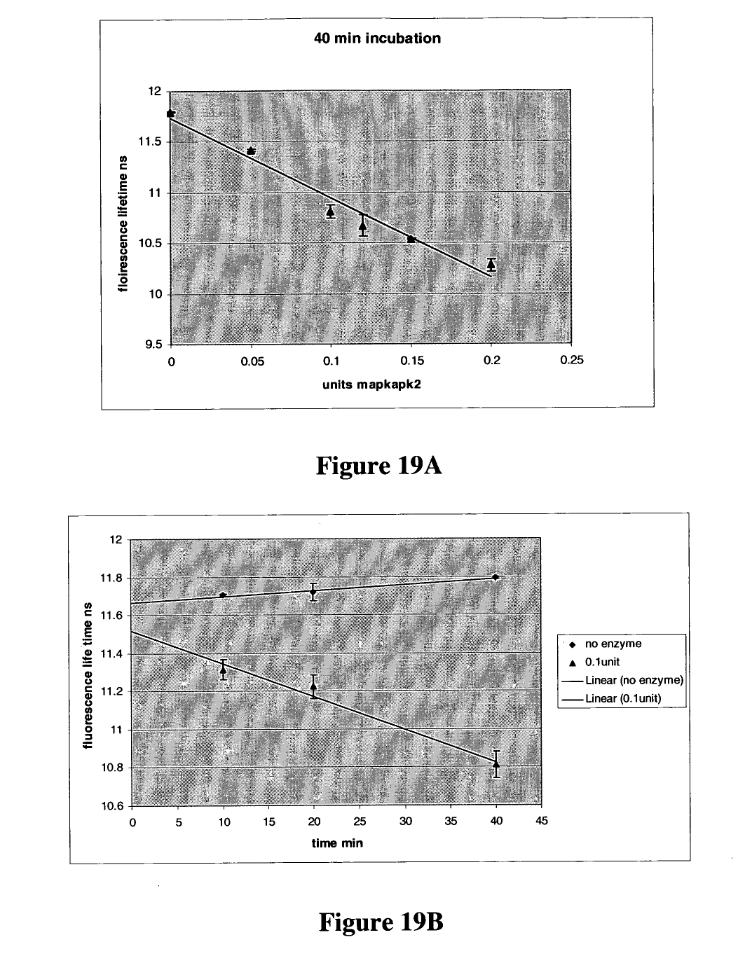

The results of the protein dependence assay and time course are shown in FIGS. 19A and B respectively.

### Experiment 11

The MAPKAP K2 assay was also performed using a substrate peptide labelled at the C terminus LNRTLSVAK. Again the peptide was N-acetylated and the label was on the lysine side chain. This alternative substrate gave data almost identical to that shown for the N terminally labelled substrate.

### Experiment 12

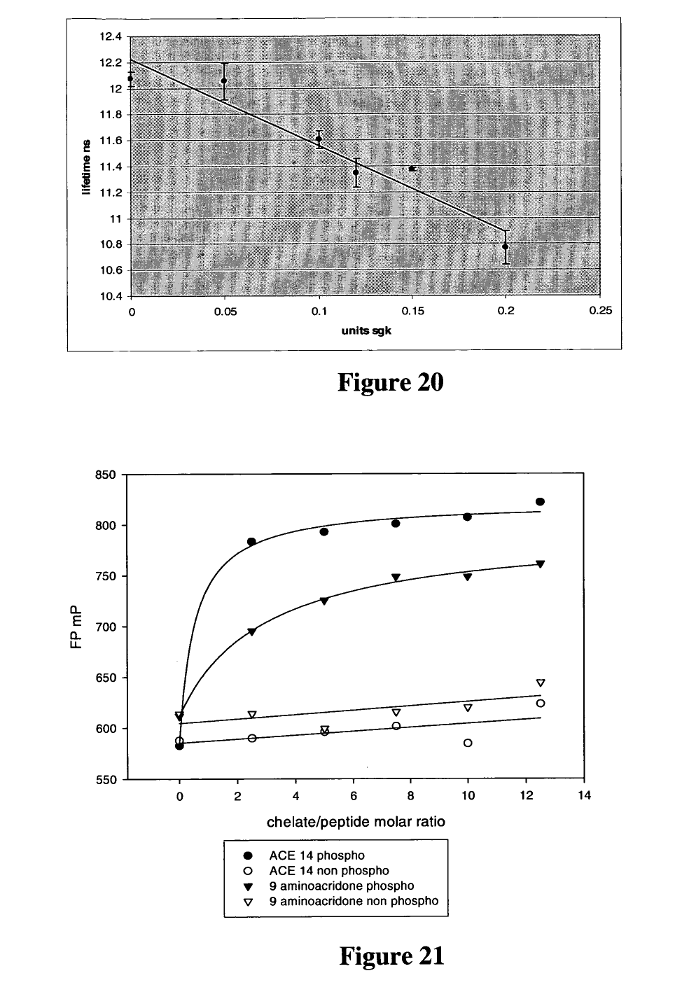

The results of a similar assay for SGK1 using Crosstide as a substrate are shown by way of the data presented in FIG. 20 from which it may be noted that using Crosstide as substrate again gives a linear dependence on protein concentration.

### Experiment 13

A BSA iron chelate was prepared as follows: BSA (Fraction V from Sigma Chemicals Ltd; 30 mg) was dissolved in 2 ml of 50 mM sodium bicarbonate buffer. Diethylenetriaminepentaacetic acid anhydride (DTPA; 90 mg) was dissolved in acidified DMSO. The BSA was labelled by the addition of 63 μl (16 μmoles) of the DTPA solution and incubation overnight at 4° C. The 30 mg of BSA contained 22.3 μmoles of available lysine residues giving an average labelling of 20 chelate molecules per BSA molecule. The labelled BSA was purified by gel filtration chromatography in acetate buffer at pH 3.0. Iron (III) was then added to the purified BSA in a solution of 50% acetic acid (0.2 ml of 100 mM Fe(III) perchlorate). The BSA iron chelate was then re-purified by gel filtration to remove the excess iron. The purified BSA iron chelate was then neutralised and protein concentration determined by Bradford assay.

To test the ability of the BSA iron chelate to bind labelled phosphopeptide increasing concentrations of chelate were incubated with 20 pmol of labelled peptides as detailed below.

The 4 peptides tested were phosphorylated and non-phosphorylated Crosstide labelled with either ACE 14 or 9-aminoacridone. The peptides were mixed with increasing concentrations of BSA chelate and fluorescence polarization measured. The results are shown in FIG. 21 in which increasing mP values indicate increased binding of the dye labelled peptide.

The results shown in FIG. 21 show that the phosphopeptide interacts with the chelate and the non-phosphorylated does not.

### Experiment 14

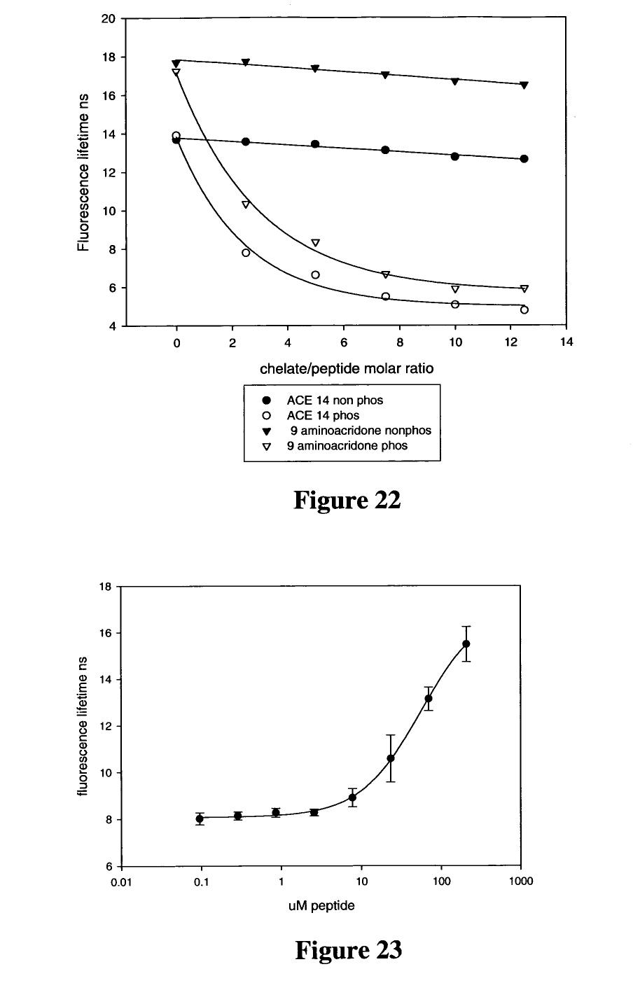

Experiment 13 was repeated but lifetime measurements were made instead of polarization. The results are shown in FIG. 22, which clearly shows a reduction in lifetime on binding to the chelate, which is dependent on the phosphorylation of the peptide.

### Experiment 15

In order to be of use in an assay the observed quenching must be reversible by other phosphorylated peptides. Initial experiments were carried out at decreasing pH from pH 5 to pH 1 which indicated that the optimum pH for displacement was about 2 to 4.5, in particular about pH 4. Therefore the subsequent experiments were conducted in acetate buffer at pH 4.

Firstly, a fixed amount of chelate complexed with 9-aminoacridone labelled Crosstide (sensor complex) was incubated with increasing concentrations of a phosphorylated hexapeptide and changes in fluorescence lifetime measured. The sensor complex was composed of BSA iron chelate and labelled peptide in a molar ratio of 10:1 at a concentration of 5 μM.

In the results shown in FIG. 23 the chelate was used at a concentration of 0.5 μM in a final volume of 100 μl containing phosphorylated hexapeptide at the concentrations shown.

The results shown in FIG. 23 demonstrate that the unlabelled phosphopeptide is capable of displacing the labelled phosphopeptide from the BSA iron chelate and generating an increasing fluorescence lifetime.

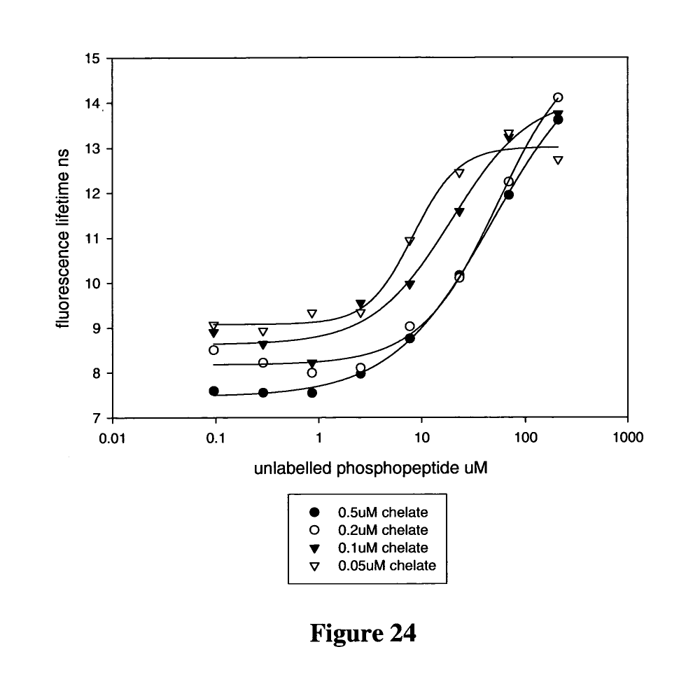

This displacement curve is somewhat too far to the right for use in a displacement assay with an IC₅₀ of 55 μM. The experiment was repeated varying the concentration of the sensor complex as shown in FIG. 24.

As may be seen from FIG. 24, decreasing the sensor concentration shifts the curve to the left. The lowest IC₅₀ using 0.05 μM sensor complex was 8.3 μM which would be useable in a displacement assay. The IC₅₀ values for the above curves are tabulated below.

| sensor μM | IC₅₀ μM |
|---|---|
| 0.05 | 8.33 |
| 0.1 | 19.44 |
| 0.2 | 53.7 |
| 0.5 | 55 |

It is noted that the sequence of the peptide will contribute to the affinity of any given peptide with, in general, longer peptides having a higher affinity. Therefore there exists a great deal of latitude in the affinity of the complex of the fluorescently labelled compound, where a peptide, with the protein, depending on the relative size of the sensor peptide to the substrate used in an assay situation. This sensor complex also has the potential to be used in situations where the kinase being assayed will only use another kinase protein or large protein as substrate.

### Experiment 16

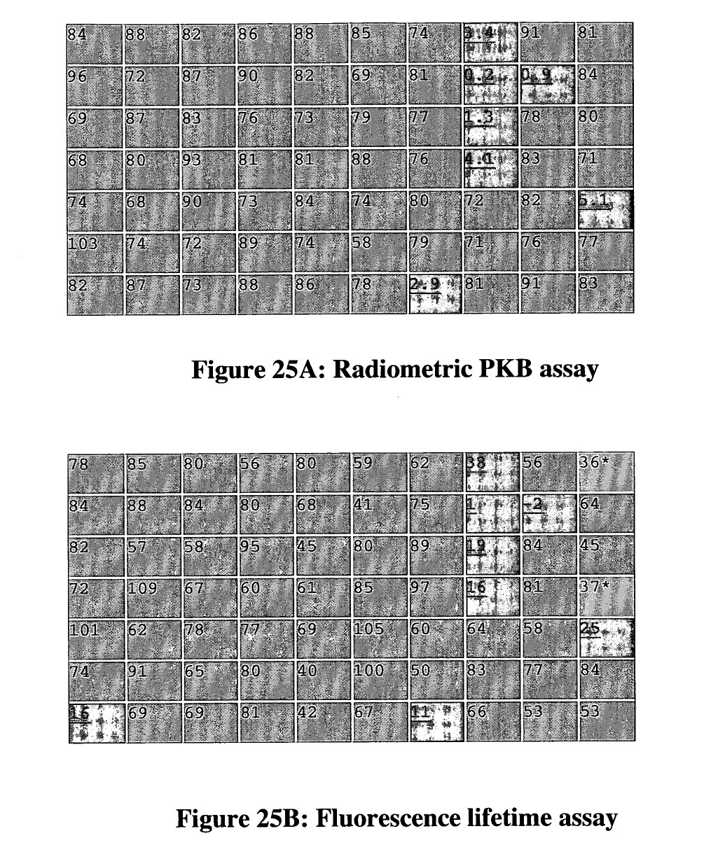

In order to test the efficacy of the assay in a screening situation a comparison of a radiometric PKB assay and fluorescence lifetime assay was carried out using a compound series sent for screening by a pharmaceutical company. In FIG. 25A underlined data denotes a significant inhibition of activity scored as a hit. The data is presented as % activity of control uninhibited reactions.

FIG. 25B shows that all hits detected by the radiometric assay are also detected by the lifetime assay. The lifetime assay is also detecting 3 other hits (the asterisked data); this could be due to liquid handling errors in this case since the radiometric assay was done robotically whereas the lifetime assay was done manually. These results indicate that with improvements in liquid handling and/or other optimisation routine to those skilled in the art the assay is effective as a screening procedure.

### Experiment 17

Additional measurements were made on four kinases (PKA, PKB, MAPKAPK2 and CHEK1) at concentrations of 0.1 U/5 ml and 0.2 U/5 ml.

Assay conditions were 0.5 mM ATP; 5 mM MgCl₂; 50 mM Tris pH 7; 10/30 μM peptide; 0.1/0.2μ enzyme per assay and 8 mM Fe (III)/PMA chelate (all concentrations final).

The measured time courses show early saturation of the system (after 5 min). The average lifetime fell from 12.5 ns to ca 9 ns.

Measurements were done in triplicate and analysed using Global Fit with two lifetimes, the unphosphorylated long lifetime of 12.5 ns and a linked shorter lifetime. The analysis of the saturated part of the time course for the 0.1 U concentration is summarised below:—

| | Short Lifetime | | | |
|---|---|---|---|---|
| Assay | ns | A₁ | A₂ | A₁/A₁ + A₂ |
| CHEK1 Pep3 | 5.07 | 3973 | 2873 | 0.58 |
| MAPKAP Pep3 | 5.23 | 3925 | 2670 | 0.595 |
| PKA Pep2 | 3.234 | 3355 | 3123 | 0.51 |
| PKB Pep1 | 3.721 | 3536 | 3132 | 0.53 |

A skilled person will appreciate that variations of the disclosed arrangements are possible without departing from the invention. For example, the fluorescence lifetime measurements and analyses could be applied to numerous different assays. Its effectiveness has, for example, been successfully demonstrated for the OMNIA™ kit Serene/Thrionine Peptide 1 produced by Invitrogen.

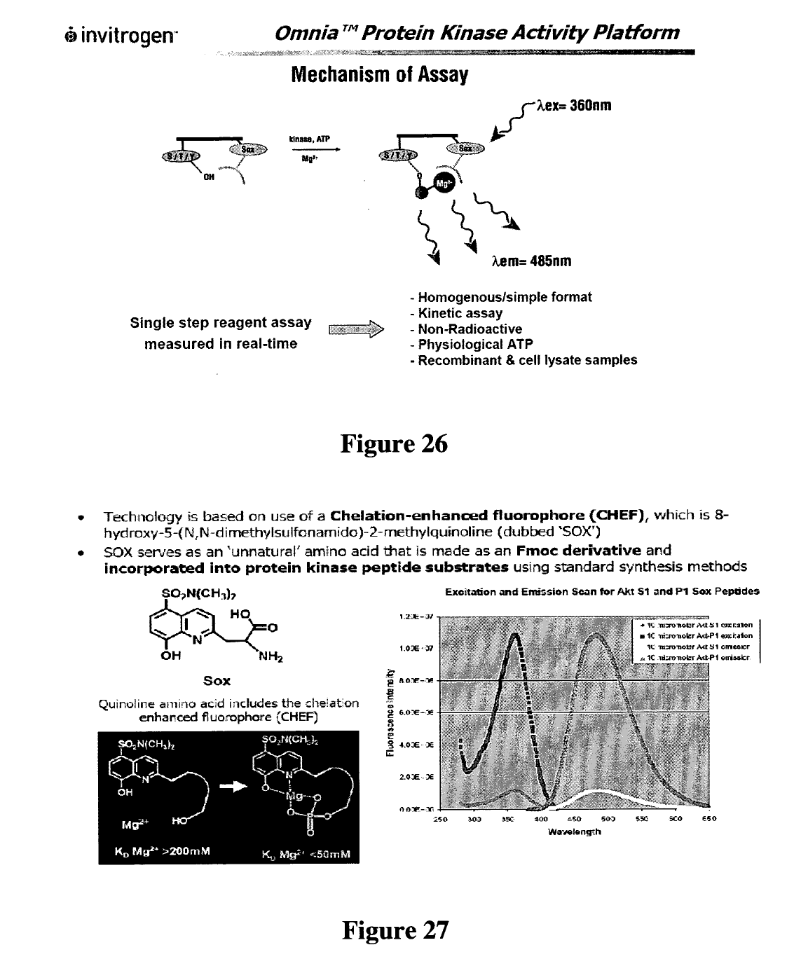

The operation of the Invitrogen assay is illustrated in FIG. 26. The assay is based on the use of an unnatural amino acid, SOX, which can be inserted in peptide substrate. SOX emission is greatly affected by phosphorylation of the substrate. A structure of SOX and the principle of its fluorescence modulation are shown in FIG. 27. When the phospho-group is phosphorylated, Mg²⁺ ion can produce a high affinity coordination complex with SOX and the phospho-group as shown. The interaction of magnesium ion with 8-hydroxy-5(N,N-dimethylsulfonamido)-2-methylquinoline significantly increases its quantum yield mostly by changing the lifetime of the SOX.

A titration of both phosphorylated and unphosphorylated substrates with magnesium was carried out in the range 0-50 mM MgCl₂. At low concentration of magnesium, there was little change to the fluorescence kinetics of both phosphorylated and unphosphorylated substrates, while at high concentrations both substrates were fully saturated and their decays were similar (i.e. unquenched). The range of magnesium concentration where significant differences in the emission of phosphorylated and unphoshorylated substrates were observed is 5-30 mM. To explore this range further, two titrations were made using different proportions of phosphorylated and unphosphorylated substrates in a PBS buffer pH7.5 (ca 1 μM) containing 25 mM and 15 mM MgCl₂ respectively.

Eleven samples were made to reflect a progression of the assay.

| Sample | % NP | % PP |
|---|---|---|
| 1 | 0 | 100 |
| 2 | 10 | 90 |
| 3 | 20 | 80 |
| 4 | 30 | 70 |
| 5 | 40 | 60 |
| 6 | 50 | 50 |
| 7 | 60 | 40 |
| 8 | 70 | 30 |
| 9 | 80 | 20 |
| 10 | 90 | 10 |
| 11 | 100 | 0 |

The decay of each sample was measured using a FLS920 spectrometer of the general form of FIG. 1 with picosecond diode laser excitation (375 nm, 0.5 MHz repetition rate) and analysed globally using the FAST software using the three-exponential model described above.

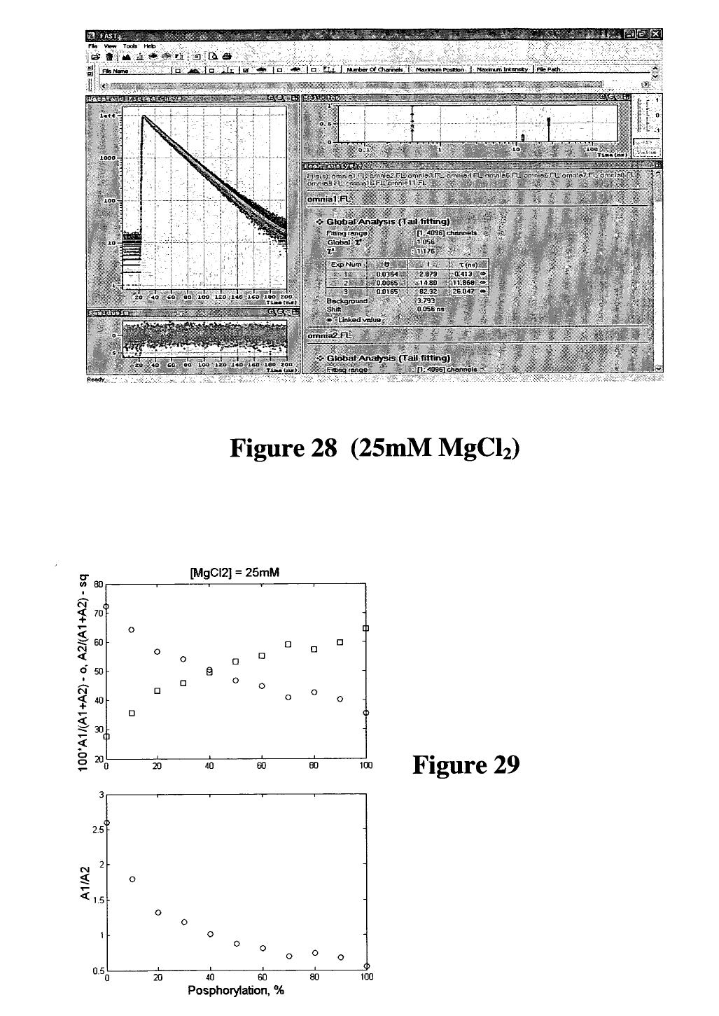

Results for 25 mM MgCl₂ Concentration are shown in FIG. 28. The eleven time courses were analysed globally for three linked lifetimes. The showed three lifetimes of 0.4 ns, 11.9 and 26.0 with amplitudes B1, B2 and B2 respectively, which varied, for different proportions of unphosphorylated and phosphorylated substrates. The 0% phosphorylated sample exhibited mostly the first and the second lifetime components (0.4 ns and 11.9 ns). The 100% phosphorylated sample exhibited both these two components, as well as a long lifetime component (26 ns). The presence of the short lifetime components in emission of the 100% phosphorylated substrate is apparently due to a limited affinity of Mg²⁺ for this substrate. Concentration dependencies of the fast and slow component's fractions f1=(B1+B2)/(B1+B2+B3) and f2=B3/(B1+B2+B3) parameters are shown in FIG. 29.

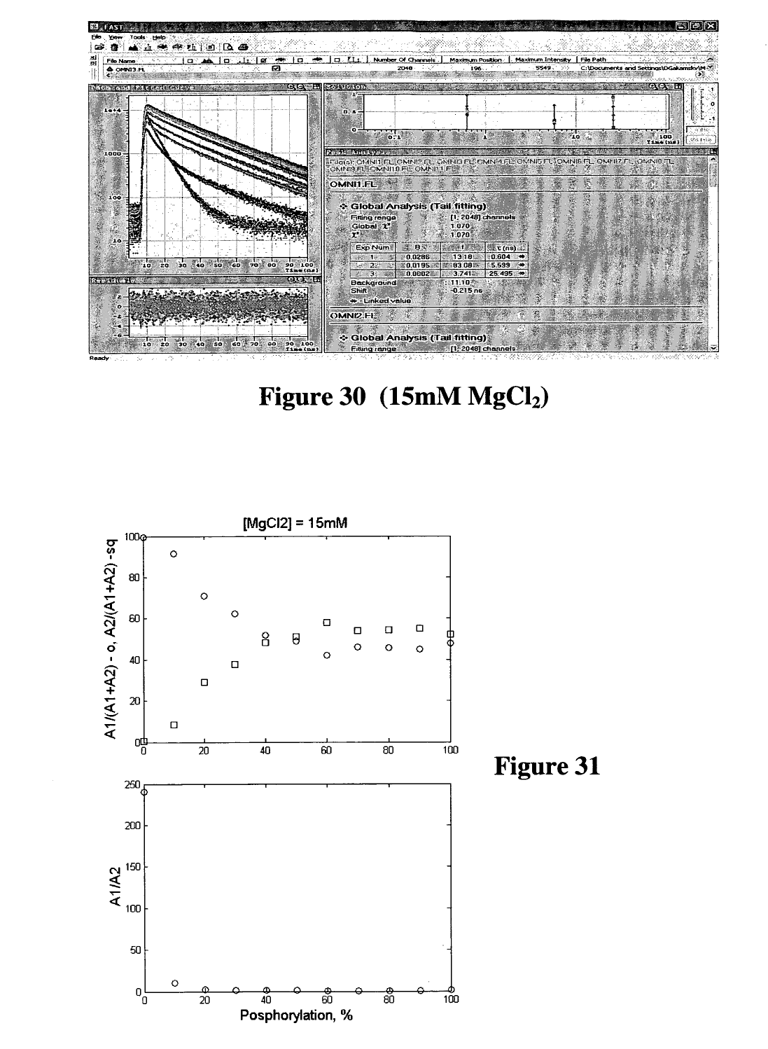

Results for 15 mM MgCl₂ Concentration are shown in FIG. 30. The entire range of phosphorylation were assessed with. Again, the 11 time courses were analysed globally for two linked lifetimes. The analysis showed three lifetimes of 0.6 ns, 5.6 ns and 25.5 ns with amplitudes B1, B2 and B3 respectively, which varied, for different proportions of unphosphorylated and phosphorylated substrates. Concentration dependences of the fast and slow component's fractions f1=(B1+B2)/(B1+B2+B3) and f2=B2/(B1+B2+B3) parameters are shown in FIG. 31.

The lower concentration of MgCl₂ (15 mM) allows a more sensitive assessment in the low phosphorylation range (0-30%). This is of importance for design of kinase inhibition assays for HTS. Lifetime analysis of SOX fluorescence (f1 and f2 parameters) provides quantitative information about the substrate phosphorylation. Their concentration dependences are saturated faster at 15 mM than at 25 mM MgCl₂. Hence, the substrate phosphorylation can be quantified in the 0-30% at 15 mM and in the 0-100% substrate phosphorylation range at 25 mM. Sensitivity to detection of substrate phosphorylation is higher at 15 mM than at 25 mM MgCl₂. Sensitivity can be enhanced by monitoring the ratio of f1/f2.

The results described with reference to FIGS. 26 to 31 demonstrate the applicability of the present invention to a range of different assays. Accordingly, the above description of a specific embodiment is made by way of example only and not for the purposes of limitation. It will be clear to the skilled person that minor modifications may be made without significant changes to the operation described.

### Experiment 18

This shows examples of Kₘ estimation using the 3 generic substrate peptides Pep1, Pep2 and Pep3 labelled with 9-amino acridine. In order to exemplify the assays Kₘ values were determined for each of the generic peptides using an appropriate kinase (see the three examples shown below each using one of the three generic substrates).

Initial experiments were carried out to determine concentrations of enzyme that would give linear time courses to allow the calculation of initial rates of reaction.

Km Calculation using MSK-1 as an example.

Enzyme stock concentration 935 units/mL

Peptide 1 as substrate

Reagent Stock Concentrations

### 1 M Tris (pH 7.0)

### 5 M NaCl₂

### 1 M DTT

### 1 M MgCl₂

10 mg mL⁻¹ BSA

### 5 mM ATP

### Generic Peptide 1 688 μM

100 mM phenylmalonic acid in water
100 mM perchlorate in glacial acetic acid

### Enzyme Buffer

### 50 mM Tris (pH 7.0)

### 150 mM NaCl

### 1 mM DTT

### 5 mM MgCl₂

1 mg mL⁻¹ BSA

### Chelate Solution (Prepared on the Day)

800 μL phenylmalonic acid solution
800 μL iron perchlorate solution
1.7 mL of water
1.7 mL of glacial acetic acid

### Assay

The assays were carried out in triplicate in polypropylene round-bottomed 96-well plates. Stock MSK-1 was diluted in enzyme buffer to give a concentration of 800 milliunits of enzyme per mL and 80 μL added to the required number of wells of the 96 well plate. Substrate peptide 1 diluted in assay mix to give concentrations of 2, 4, 10, 20, 40 and 60 μM and 100 μL was added to the enzyme solution in the wells. The reactions were initiated by addition of 20 μL 5 mM ATP (0.5 mM final concentration). This results in a series of 200 μL assays at final substrate concentrations of 1, 2, 5, 10, 20 and 30 μM. The control reaction was an enzyme-free solution containing 10 μM peptide 1 in enzyme buffer. At time points 0, 5, 10, 20 and 30 minutes after initiation of the assays 25 μl, of each reaction mixture was removed and added to a black flat-bottomed Greiner plate containing 25 μL of chelate solution per well. After collection of all time points the plate was read in a Nano Taurus plate reader and the average lifetime of the labelled peptide in each well was determined as shown in Table 2.

TABLE 2
Fluorescence lifetime

| | Pep μM | | | | | | |
|---|---|---|---|---|---|---|---|
| Time | 0 | 1 | 2 | 5 | 10 | 20 | 30 |
| 0 | 15.38945 | 15.14835 | 15.2825 | 15.30955 | 15.42401 | 15.17302 | 15.07404 |
| 5 | 15.39128 | 14.35869 | 14.76012 | 14.87283 | 14.95656 | 15.04799 | 14.86548 |
| 10 | 15.3711 | 13.11512 | 13.57122 | 14.15885 | 14.61103 | 14.78301 | 14.67659 |
| 20 | 15.32862 | 11.61246 | 11.7419 | 12.47883 | 13.53876 | 14.26393 | 14.26857 |
| 30 | 15.25233 | 11.35924 | 11.44004 | 11.36181 | 12.03632 | 13.36471 | 13.68857 |

Using a standard curve constructed by mixing known amounts of phospho and de-phospho peptide the lifetime data in Table 2 can be recalculated to give the % phospho peptide present in each assay (Table 3a) and since the concentration of substrate is known the amount of product formed can be calculated (Table 3b). The data can be corrected for controls and zero time points as (Table 3c) and the time course data plotted to give initial rates of reaction.

TABLE 3a
% substrate conversion

| | Pep μM | | | | | | |
|---|---|---|---|---|---|---|---|
| Time | 0 | 1 | 2 | 5 | 10 | 20 | 30 |
| 0 | 5.194573 | 15.33233 | 9.86606 | 8.71258 | 3.618347 | 14.35682 | 18.19548 |
| 5 | 5.112022 | 41.41262 | 29.22815 | 25.4455 | 22.51146 | 19.17405 | 25.69783 |
| 10 | 6.018056 | 71.05966 | 61.22843 | 46.8731 | 33.96885 | 28.47437 | 31.91878 |
| 20 | 7.888211 | 98.52576 | 96.36997 | 83.43136 | 61.96078 | 44.04515 | 43.91819 |
| 30 | 11.13129 | 102.6601 | 101.3522 | 102.6187 | 91.34673 | 65.79789 | 58.53333 |

TABLE 3b
μmoles product

| | Pep μM | | | | | | |
|---|---|---|---|---|---|---|---|
| Time | 0 | 1 | 2 | 5 | 10 | 20 | 30 |
| 0 | 0.051946 | 0.153323 | 0.098661 | 0.087126 | 0.036183 | 0.143568 | 0.181955 |
| 5 | 0.05112 | 0.414126 | 0.292282 | 0.254455 | 0.225115 | 0.191741 | 0.256978 |
| 10 | 0.060181 | 0.710597 | 0.612284 | 0.468731 | 0.339689 | 0.284744 | 0.319188 |
| 20 | 0.078882 | 0.985258 | 0.9637 | 0.834314 | 0.619608 | 0.440452 | 0.439182 |
| 30 | 0.111313 | 1.026601 | 1.013522 | 1.026187 | 0.913467 | 0.657979 | 0.585333 |

TABLE 3c
time zero and control subtracted

| | Pep μM | | | | | | |
|---|---|---|---|---|---|---|---|
| Time | 0 | 1 | 2 | 5 | 10 | 20 | 30 |
| 0 | 0 | 0 | 0 | 0 | 0 | 0 | 0 |
| 5 | −0.00083 | 0.260803 | 0.387242 | 0.836646 | 1.889311 | 0.963447 | 2.250706 |
| 10 | 0.008235 | 0.557273 | 1.027247 | 1.908026 | 3.035051 | 2.82351 | 4.116992 |
| 20 | 0.026936 | 0.831934 | 1.730078 | 3.735939 | 5.834244 | 5.937666 | 7.716813 |
| 30 | 0.059367 | 0.873278 | 1.829722 | 4.695304 | 8.772839 | 10.28821 | 12.10135 |

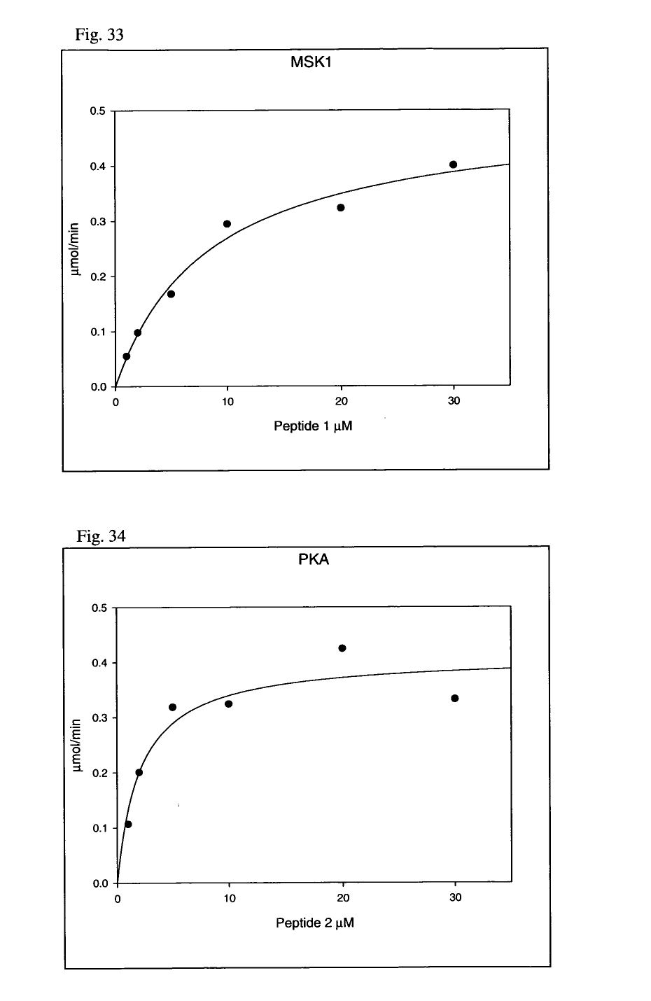

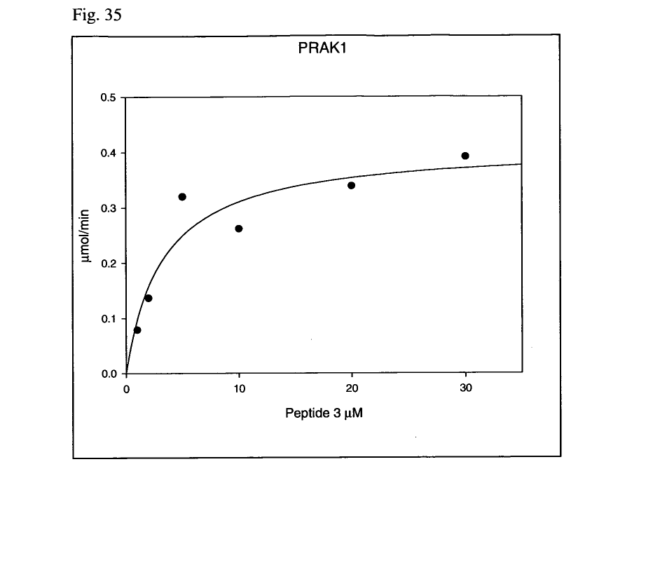

These initial rates were then plotted against substrate concentration (FIG. 33) and the hyperbolic curve fitted by non linear regression to obtain a Kₘ value. The calculated Kₘ is 8.5 μM which compares to a value of 2 μM as determined by radiometric assay. This difference is probably due to the presence of the dye on the substrate peptide changing the affinity of the substrate for the enzyme. The generic peptides 2 and 3 were also assayed as described above using the enzymes PKA and PRAK1 respectively. The data obtained are shown in FIGS. 34 and 35. The Kₘ values for several kinases, obtained as described above, using peptides 1, 2 and 3 labelled with Ace 14 or 9-amino acridine (9AA) in comparison to values obtained by radio-metric assay are shown in Table 4.

TABLE 4

| | | Ace14 | | 9AA |
|---|---|---|---|---|
| Peptide | Enzyme | Km | Radio-metric | Km |
| 3 | CHK1 | 9 | 5 | 3 |
| 2 | PKA | 3 | 30 | 7 |
| 3 | MAPKAPK2 | 18 | 5 | 1 |
| 1 | p70s6K | | 6 | 29 |
| 1 | PKBα | | 2 | 4 |
| 3 | PRAK1 | | 40 | 3 |
| 1 | SGK1 | | 4 | 2 |
| 1 | MSK1 | | 2 | 8 |

Some values are in close agreement where as some show higher or lower affinity for the dye labelled peptides. This is to be expected considering the addition of a large relatively hydrophobic moiety to the peptide sequence which in some cases may increase and others decrease interaction with the enzymes.

### Experiment 19

### PKA-PKB Inhibitor Test Screen

In order to carry out a comparison of fluorescence lifetime assay and radiometric assay in an inhibitor screen plates of test compounds which had already been screened by radiometric assay were obtained and re-assayed by fluorescence lifetime.

In order to carry out a screening assay the reaction is advantageously in the linear range of activity. In order to linearise the assays activity time courses were conducted with varying concentrations of enzyme in order to verify conditions giving linear rates of activity under the screening format conditions.

### Assay PKA

A solution (25 μL) containing peptide 2 (10 μM) and PKA (5 milliunits) was added to each well containing inhibitor. The assay was initiated by the addition of 1 mM ATP (25 μL). Each plate also contained a row of positive control wells containing no inhibitor and a row of ATP-free negative controls. The assay was stopped after 20 minutes by adding 25 μL of assay mixture to a black 96-well flat-bottomed plate with 25 μL of chelate solution per well.

### Assay PKB

A solution (25 μL) containing peptide 1 (10 μM) and PKB (40 milliunits) was added to each well containing inhibitor. The assay was initiated by the addition of 1 mM ATP (25 μL). Each plate also contained a row of positive control wells containing no inhibitor and a row of ATP-free negative controls. The assay was stopped after 20 minutes by adding 25 μL of assay mixture to a black 96-well flat-bottomed plate with 25 μL of chelate solution per well.

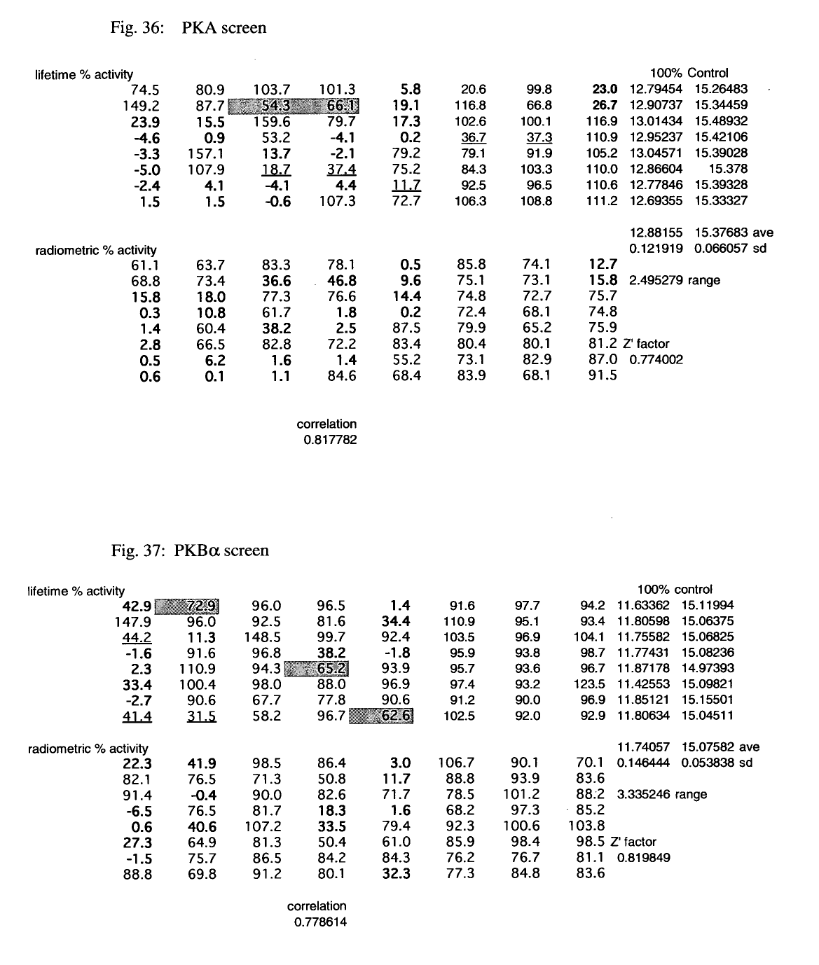

The results of the screen are shown in FIGS. 36 and 37. Inhibitors identified by the radiometric assay (greater than 50% reduction in activity) are shown by the values for percentage activity presented in bold type, as are the lifetime assays. Percentage activities represented with a shaded background indicate inhibition detected by lifetime but not as potent as determined by the radiometric assay. Numbers that are underlined are of percentage activities where the compounds showing inhibition that were not detected by radiometric assay.

In the case of the PKA assay compounds defined as inhibitor hits by fluorescence lifetime match 91% with the radiometric assay. This increases to 100% if the compound showing partial inhibition (percentage activities represented with a shaded background) are included. The lifetime assay also identified 5 compounds showing inhibitory activity that were not detected by the radiometric assay.

In the case of the PKB assay compounds defined as inhibitor hits by fluorescence lifetime match 71% with the radiometric assay. This increases to 91% if the compound showing partial inhibition (percentage activities represented with a shaded background) are included. The lifetime assay also identified 3 compounds (percentage activities shown underlined) showing inhibitory activity that were not detected by the radiometric assay.

1. A method for determining a degree of phosphorylation of a substrate, using a fluorescence probe that acts alone or with another material and has a lifetime that varies when in proximity to a phosphate, the method comprising:
causing the fluorescence probe to fluoresce;
measuring a time response of the fluorescence, and
analyzing the fluorescence time response to identify a fluorescence component having a lifetime associated with phosphorylated substrate and a fluorescence component having a lifetime associated with un-phosphorylated substrate.

2. The method as claimed in claim 1 wherein analyzing the fluorescence time response, I(t), involves using a model function constructed as a linear combination of emission functions of unphosphorylated, I₁(t), and phosphorylated, I₂(t) substrates: I(t)=A₁I₁(t)+A₂I₂(t), where A₁ and A₂ are amplitude coefficients of phosphorylated and un-phosphorylated substrates respectively.

3. The method as claimed in claim 1 further comprising fitting the fluorescence time response to the model to discriminate between false positive or negative hits based on the quality of the fit to the model.

4. The method of claim 1 further comprising applying the substrate to a kinase before causing the fluorescence probe to fluoresce and using the step of analysing to determine phosphorylation of the substrate.

5. The method of claim 1 further comprising applying the substrate to a phosphatase before causing the fluorescence probe to fluoresce and using the step of analyzing to determine dephosphorylation of the substrate.

6. A method for investigating the phosphorylation of a fluorescently labeled test compound by a kinase, said method comprising contacting said test compound with said kinase and a quenching moiety and measuring the resultant fluorescence, and said quenching moiety quenching fluorescence from the test compound upon phosphorylation of the test compound.

7. The method of claim 6 wherein the kinase is a serine/threonine kinase.

8. The method of claim 6 wherein the kinase is PKA, PKB, MAPKAPK2 or CHEK1.

9. The method of claim 6 wherein the kinase is PKB.

10. The method of claim 6 wherein the kinase is PKB alpha.

11. A method for investigating the dephosphorylation of a fluorescently labeled test compound by a phosphatase, wherein said test compound comprises a phosphate moiety, said method comprising contacting said test compound with said phosphatase and a quenching moiety and measuring the resultant fluorescence, and said quenching moiety quenching fluorescence from the test compound.

12. The method of claim 11 wherein the quenching moiety is or is part of a polydentate ligand.

13. The method of claim 12 wherein the quenching moiety is provided by an aromatic- or heteroaromatic-containing polydentate ligand.

14. The method of claim 12 wherein the quenching moiety is an aromatic-containing bi- or tridentate ligand.

15. The method of claim 12 wherein the quenching moiety is coordinated to an iron (III) ion.

16. The method of claim 12 wherein the quenching moiety is comprised by a chelate formed between Fe(III) and phenylmalonic acid or between Fe(III) and 2-hydroxyacetophenone.

17. A method for investigating the activity of a kinase, said method comprising
(i) exposing the kinase to a test compound in the presence of a complex comprising a fluorescently labeled phosphorylated compound and quenching moiety, wherein said quenching moiety quenches fluorescence of the fluorescent label in said complex; and
(ii) measuring the resultant fluorescence.

18. The method of claim 17 wherein the kinase is a serine/threonine kinase.

19. A method for investigating the activity of phosphatase, said method comprising
(i) exposing the phosphatase to a test compound wherein some of said test compound is present in a complex comprising said test compound and quenching moiety, in the presence of a fluorescently labeled phosphorylated compound wherein said quenching moiety quenches fluorescence of the fluorescent label upon complexation of the protein to the fluorescently labeled phosphorylated compound; and
(ii) measuring the resultant fluorescence.

20. The method as claimed in claim 19 wherein said quenching moiety/protein is modified to comprise one or more moieties that serve to interact with, or coordinate to, a phosphate group.

21. The method as claimed in claim 20 wherein said moieties are iron (III) ions.

22. The method of claim 20 wherein said moieties are chelated to a protein-bound coordinating ligand.

23. The method of claim 22 wherein said protein-bound coordinating ligand is polydentate.

24. The method of claim 23 wherein said protein-bound polydentate coordinating ligand is diethylenetriamine pentaacetic acid.

25. The method of claim 19 wherein the fluorescent label is a fluorescence lifetime label.

26. The method of claim 25 wherein the fluorescence lifetime label has a fluorescent lifetime in the range of 12 to 25 ns.

27. The method of claim 19 wherein the fluorescent label is an acridone, quinacridone or acridine.

28. The method of claim 19 wherein the fluorescent label is O-(N-Succinimidyl)-6-(9-oxo-9H-acrodin-10yl) hexanoate.

29. The method of claim 19 wherein the fluorescence measured is fluorescence intensity or fluorescent lifetime.

30. The method of claim 19 wherein the fluorescence measured is fluorescence lifetime.

31. The method of claim 30 wherein the fluorescence lifetime is measured by a method as defined in claim 1.

32. The method of claim 19 wherein the test compound is a protein, a peptide or a lipid.

33. The method of claim 32 wherein the test compound is a protein or a peptide.

34. The method of claim 32 wherein the test compound is a peptide.

35. The method of claim 33 wherein the test compound has a phosphate moiety or a moiety susceptible to phosphorylation which moiety is part of an amino acid 10 or fewer positions away from the amino acid to which the fluorescent label is attached.

36. The method of claim 19 wherein the fluorescent label is attached to a terminal amino acid.

37. The method of claim 19 wherein the test compound is attached to a transport peptide.

38. The method of claim 37 wherein the investigating is of a kinase activity in a crude cell lysate or a whole cell.

39. The method of claim 19 wherein the test compound is bound to a solid support by a linker group.

40. The method of claim 19 wherein the test compound is capable of being processed by said kinase or said phosphatase and said measuring permits the activity of said kinase or said phosphatase to be determined.

41. The method of claim 19 wherein the test compound is known to be a substrate of said kinase or said phosphatase and the method is conducted in the presence of an additional compound.

42. The method of claim 41 when conducted with a plurality of said additional compounds.

43. The method of claim 41 wherein said method is conducted in the wells of a multiwell plate, in assay tubes or in the microchannels of a microfluidic device.

44. (canceled)

45. A kit of parts suitable for use in a method as defined in claim 6 comprising a fluorescently labeled kinase or phosphatase substrate and a quenching moiety.

46. A kit of parts suitable for use in a method as defined in claim 17 comprising a fluorescently labeled labelled phosphorylated compound and a protein with which it may be complexed.

47. The method of claim 6, wherein said resultant fluorescence is lifetime fluorescence.

48. The method of claim 11, wherein said resultant fluorescence is lifetime fluorescence.


SEQUENCE LISTING (4 sequences)

SEQ ID NO: 1 - protein, 11 aa, Artificial
  Synthetic peptide
GRPRTSSFAEG

SEQ ID NO: 2 - protein, 11 aa, Artificial
  Synthetic peptide
RARTLSFAEPG

SEQ ID NO: 3 - protein, 10 aa, Artificial
  Synthetic peptide
RRRLSFAEPG

SEQ ID NO: 4 - protein, 14 aa, Artificial
  Synthetic peptide
KKLNRTLSFFAEPG